(19) United States
(12) Patent Application Publication
(10) Pub. No.: US 2008/0089845 A1
(43) Pub. Date: Apr. 17, 2008

(54) METHODS FOR DETERMINING EFFECTIVE DOSES OF FATTY ACID AMIDE HYDROLASE INHIBITORS IN VIVO

(75) Inventors: Timothy R. Compton, Issaquah, WA (US); Jeff A. Parrott, Irvine, CA (US); Edward P. Monaghan, Rancho Santa Margarita, CA (US); Olivier Dasse, Foothill Ranch, CA (US); David Putman, Irvine, CA (US)

Correspondence Address: ORGANON USA, INC.;PATENT DEPARTMENT, 56 LIVINGSTON AVENUE, ROSELAND, NJ, 07068, US

(73) Assignee: N.V. Organon, Oss (NL)

(21) Appl. No.: 11/847,633

(22) Filed: Aug. 30, 2007

Related U.S. Application Data
(60) Provisional application No. 60/824,887, filed on Sep. 7, 2006.
(60) Provisional application No. 60/947,869, filed on Jul. 3, 2007.

(51) Int. Cl. A61K 31/423; A61K 31/245; A61K 31/41; A61P 25/18; A61P 25/24; A61P 29/00; A61P 25/28; A61P 25/22; A61K 49/00; A61K 31/4184

(52) U.S. Cl. 424/9.2; 514/367; 514/375; 514/381; 514/506

(57) ABSTRACT

Described herein is a method for determining an effective dose of a composition for inhibiting fatty acid amide hydrolase activity in vivo, by first administering to a subject a dose of a test composition, and subsequently assessing if the level of a fatty acid amide in the subject increases. Also described, is a method for optimizing therapeutic efficacy for treatment of anxiety, depression, pain, or a metabolic disorder by increasing or decreasing a dose of a fatty amide hydrolase inhibitor according to a patient's fatty acid amide levels. In addition, pharmaceutical compositions are described, which contain fatty acid amide hydrolase inhibitors effective for increasing a FAA level in a patient.

### CROSS-REFERENCE TO RELATED APPLICATIONS

This application claims priority based on U.S. Provisional Application No. 60/824,887, filed Sep. 7, 2006, the contents of which are incorporated by reference in their entirety, and from U.S. Provisional Application No. 60/947,869 filed Jul. 3, 2007.

### BACKGROUND

The fatty acid amides (FAAs) are an endogenous family of multifunctional signaling lipids that regulate pain, fear, anxiety, depression, memory, motor coordination, inflammation, and metabolism. Anandamide, one of the most well characterized FAAs, acts through cannabinoid type I receptors to confer analgesic, anxiolytic, and anti-depressant effects. Unfortunately, the therapeutic potential of anandamide is tempered by the fact that, when administered systemically, it also causes catalepsy, hypothermia, and hyperphagia, due to the ubiquitous activation of cannabinoid receptors. Further, the prolonged use of a cannabinergic agonist such as anandamide may cause addiction. An alternative approach for exploiting anandamide's therapeutic properties is to modulate its endogenous levels along with those of other FAAs.

### SUMMARY OF THE INVENTION

Assays of systemic levels of one or more FAAs can be used to determine the ability of a given dose of a test composition to increase endogenous anandamide levels. Further, assays of systemic levels of one or more FAAs can be used to determine the ability of a given dose of a test composition to inhibit fatty acid amide hydrolase activity. Further, assays of systemic levels of one or more FAAs can be used to indicate whether there is a need for a subject to receive a different subsequent dose of a fatty acid amide hydrolase inhibitor (i.e., increasing the next dose, decreasing the next dose or maintaining the next dose). In any of the above assays (or any of the methods and assays described herein), suitable FAAs include fatty acid ethanolamides with a fatty acid moiety containing 14 to 28 carbons, with 0 to 6 double bonds, such as oleoylethanolamide (OEA), palmitoylethanolamide (PEA), stearoylethanolamide (SEA) and anandamide (AEA). Other suitable FAAs include primary fatty acid amides with a fatty acid moiety containing 14 to 28 carbons, with 0 to 6 double bonds, such as oleamide. In some embodiments, the subject is suffering from a psychiatric, neurological, neurodegenerative, painful, or metabolic disorder. Also described are pharmaceutical compositions that provide a sufficient amount of an inhibitor of fatty acid amide hydrolase to increase the level of at least one FAA (e.g., oleoylethanolamide, palmitoylethanolamide, or stearoylethanolamide) by a desired amount (e.g., by at least about 30%; by at least about 50%; by at least about 70%; by at least about 100%; by at least about 150%; or by at least about 200%).

Methods are described herein for determining doses of compositions that effectively inhibit FAAH activity in vivo and which are therapeutically effective for treating conditions ameliorated by an elevated physiological level of anandamide. Pharmaceutical compositions containing FAAH inhibitors are also described.

Accordingly, one aspect described herein, relates to a method for determining an effective dose of a composition for increasing endogenous levels of anandamide. A level of a FAA other than anandamide (e.g., oleoylethanolamide, palmitoylethanolamide, or stearoylethanolamide) is determined in a biological sample (e.g., plasma, cerebrospinal fluid, saliva, urine) obtained from a subject (e.g., a non-human primate or a human) at a first time point. Afterwards, the subject is administered (e.g., orally) a dose of a test composition (e.g., an alkylcarbamic acid aryl ester). For example, the test composition can be KDS-4103. A biological sample is then obtained from the subject at a second time point, and a level of the FAA is determined. The dose of the test composition is considered effective when the FAA level in the biological sample obtained at the second time point is determined to be greater than in the biological sample obtained at the first point (e.g., by at least about 30%; by at least about 50%; by at least about 70%; by at least about 100%; by at least about 150%; or by at least about 200%) or the FAA level at the second time point is determined to be at least about 90% of its saturation value. In other embodiments, the need for two time-points can be eliminated by relying on a statistically-relevant pre-determined level of a FAA (i.e., a level that is considered normal for a particular population). In some embodiments, the subject is suffering from a psychiatric, neurological, neurodegenerative, painful, or metabolic disorder. In one embodiment, a diagnostic evaluation of a subject suffering from one of the foregoing disorders is performed before and after administering a dose of a test composition. In some embodiments the level of the FAA is determined using chromatographic techniques; in other embodiments, the level of the FAA is determined using mass spectrometric techniques; in other embodiments, the level of the FAA is determined using spectrophotometric techniques; in other embodiments, the level of the FAA is determined using a biological assay; in other embodiments, the level of the FAA is determined using a combination of the aforementioned techniques. In any of the foregoing embodiments, assays for FAA levels can be partly or fully automated for high throughput.

In a related aspect, an effective dose of a composition for inhibiting FAAH in vivo is determined by obtaining a biological sample from a subject that has been administered a test composition, determining a FAA level (e.g., oleoylethanolamide, palmitoylethanolamide, or stearoylethanolamide) in the sample, and then comparing the FAA level to a pre-determined value. The dose of the test composition is concluded to be effective if the FAA level is greater than the pre-determined value (e.g., by at least about 30%; by at least about 50%; by at least about 70%; by at least about 100%; by at least about 150%; or by at least about 200%).

Another aspect relates to a method for optimizing therapeutic efficacy for treatment of anxiety, depression, pain, or a metabolic disorder by administering to a subject in need thereof a drug providing an alkylcarbamic acid aryl ester of Formula (I): 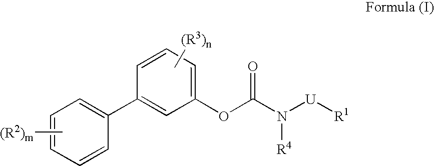
where:
R¹ is selected from among C₁-C₈ alkyl, C₁-C₄alkyl-(C₃-C₈cycloalkyl), and C₃-C₈ cycloalkyl (e.g., cyclohexyl); R⁴ is H or alkyl;
U is a bond or CH₂;
R² and R³ are each independently selected from among H, C₁-C₄ alkyl, C₂-C₄ alkenyl, C₂-C₄ alkynyl, C₃-C₆ cycloalkyl, C₁-C₄alkyl-(C₃-C₆cycloalkyl), aryl, substituted aryl, arylalkyl, —C(O)R^{A}, hydroxy-(C₁-C₆ alkyl), amino-(C₁-C₆ alkyl), —CH₂—NR^{A}R^{B}, —O—(C₁-C₄), aryloxy, halo, C₁-C₆-haloalkyl, cyano, hydroxy, nitro, amino, —C(O)NR^{A}R^{B}, —ONR^{A}R^{B}, —O—C(O)NR^{A}R^{B}, —SO₂NR^{A}R^{B};
R^{A} and R^{B} are each independently selected from among hydrogen, C₁-C₆ alkyl, and C₃-C₆ cycloalkyl; and m and n are each independently 0-3.

After administering the drug dose, a level of a FAA is determined in a biological sample from the subject. A level of FAA about 50% less than a pre-determined value (e.g., the subject's pre-determined desired level, or a statistically-relevant level determined for a population), indicates that the drug dose needs to be increased. In one embodiment, the drug provides KDS-4103:

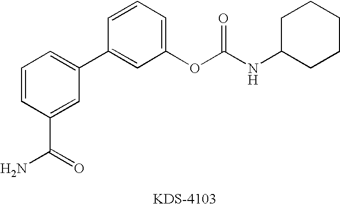

In other embodiments, the drug provides an alkylcarbamic acid aryl ester of Formula (II):

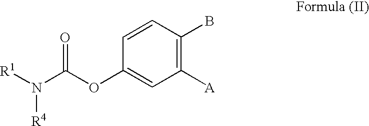

where R¹ is substituted or unsubstituted C₃-C₈ alkyl (including linear, branched, cyclic alkyl groups and combinations thereof); R⁴ is H or alkyl; and A and B are selected from:
(i) one of A or B is C(O)-alkyl, and the other is H, alkyl, heteroalkyl; A and B can combine into a non-aromatic cyclic group; A and B can be substituted;
(ii) A and B together form an optionally substituted heteroaromatic group; A and/or B are N, S, or O; or
(iii) one of A or B is L-X-G; the other is H, alkyl; L is optionally substituted alkyl or heteroalkyl; X is a bond, O, —C(═O), —CR₉(OR₉), S, —S(═O), —S(═O)₂, —NR₉, —NR₉C(O), —C(O)NR₉, —S(═O)₂NR₉—, —NR₉S(═O)₂, —OC(O)NR₉—, —NR₉C(O)O—, —NR₉C(O)NR₉—, —NR₉C(═NR₁₀)NR₉—, —NR₉C(═NR₁₀)—, —C(═NR₁₀)NR₉—, —OC(═NR₁₀)—, or —C(═NR₁₀)O—; G is H, tetrazolyl, —NHS(═O)₂R₈, S(═O)₂N(R₉)₂, —OR₉, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₉, —S(═O)₂R₉, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(O)O—, —NHC(O)NH—, —NHC(O)O, —O(O)CNH—, —NHC(O), —C(O)NH, —C(O)O, or —OC(O); each R₈ is independently selected from substituted or unsubstituted lower alkyl; each R₉ is independently selected from H, substituted or unsubstituted lower alkyl; and each R₁₀ is independently selected from H, —S(═O)₂R₈, —S(═O)₂NH₂—C(O)R₈, —CN, or —NO₂.

Yet another aspect relates to a pharmaceutical composition that contains an alkylcarbamic acid aryl ester of Formula I (e.g., KDS-4103), Formula II, or Formula IIa in an amount sufficient to increase the systemic level of at least one FAA (e.g., oleoylethanolamide, palmitoylethanolamide, or stearoylethanolamide) in a patient by at least about 50% for at least two hours when administered in a solid oral dosage form.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” -include plural referents unless the context clearly dictates otherwise. In this application, the use of “or” means “and/or” unless stated otherwise. Furthermore, use of the term “including” as well as other forms, such as “include”, “includes,” and “included,” is not limiting.

Unless defined otherwise, all technical and scientific terms used herein have the same meaning as is commonly understood by one of skill in the art to which the claimed subject matter belongs.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, may be branched, straight chain, or cyclic. Depending on the structure, an alkyl group can be a monoradical or a diradical (i.e., an alkylene group).

As used herein, C₁-Cₓ includes C₁-C₂, C₁-C₃ . . . C₁-Cₓ.

The “alkyl” moiety may have 1 to 10 carbon atoms (whenever it appears herein, a numerical range such as “1 to 10” refers to each integer in the given range; e.g., “1 to 10 carbon atoms” means that the alkyl group may have 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups can be substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

The term “alkenyl” refers to a type of alkyl group in which the first two atoms of the alkyl group form a double bond that is not part of an aromatic group. That is, an alkenyl group begins with the atoms —C(R)═C(R)—R, wherein R refers to the remaining portions of the alkenyl group, which may be the same or different. Non-limiting examples of an alkenyl group include —CH═CH₂, —C(CH₃)═CH₂, —CH═CHCH₃ and —C(CH₃)═CHCH₃. The alkenyl moiety may be branched, straight chain, or cyclic (in which case, it would also be known as a “cycloalkenyl” group). Depending on the structure, an alkenyl group can be a monoradical or a diradical (i.e., an alkenylene group). Alkenyl groups can be optionally substituted.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which may be the same or different. Non-limiting examples of an alkynyl group include —C≡CH, —C≡CCH₃ and —C≡CCH₂CH₃. The “R” portion of the alkynyl moiety may be branched, straight chain, or cyclic. Depending on the structure, an alkynyl group can be a monoradical or a diradical (i.e., an alkynylene group). Alkynyl groups can be optionally substituted.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, “Protective Groups in Organic Synthesis,” 3^{rd} Ed., John Wiley & Sons, New York, N.Y., 1999, which is incorporated herein by reference in its entirety.

The term “aromatic” refers to a planar ring having a delocalized π-electron system containing 4n+2 π electrons, where n is an integer. Aromatic rings can be formed by five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

The term “bond” or “single bond” refers to a chemical bond between two atoms, or two moieties when the atoms joined by the bond are considered to be part of larger substructure.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Illustrative examples of cycloalkyl groups include the following moieties:

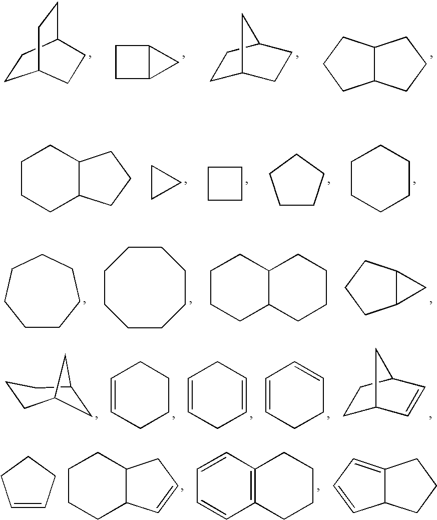

and the like. Depending on the structure, a cycloalkyl group can be a monoradical or a diradical (e.g., an cycloalkylene group).

The term “effective amount,” refers to the amount of an active FAAH inhibitor composition that is required to confer a therapeutic or cosmetic effect on the subject. A “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein, such as, a compound of Formula (I), a compound of Formula (II), or a compound of Formula (IIa), is an amount effective to achieve a desired pharmacologic effect or therapeutic improvement without undue adverse side effects. It is understood that “an effect amount” or “a therapeutically effective amount” can vary from subject to subject, due to variation in metabolism of the compound of Formula (I), a compound of Formula (II), or a compound of Formula (IIa), age, weight, general condition of the subject, the condition being treated, the severity of the condition being treated, and the judgment of the prescribing physician.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, “Protective Groups in Organic Synthesis,” 3^{rd} Ed., John Wiley & Sons, New York, N.Y., 1999.

The term “halo” or, alternatively, “halogen” or “halide” means fluoro, chloro, bromo or iodo.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

As used herein, the terms “heteroalkyl” “heteroalkenyl” and “heteroalkynyl” include optionally substituted alkyl, alkenyl and alkynyl radicals in which one or more skeletal chain atoms are selected from an atom other than carbon, e.g., oxygen, nitrogen, sulfur, silicon, phosphorus or combinations thereof.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. The polycyclic heteroaryl group may be fused or non-fused. Illustrative examples of heteroaryl groups include the following moieties:

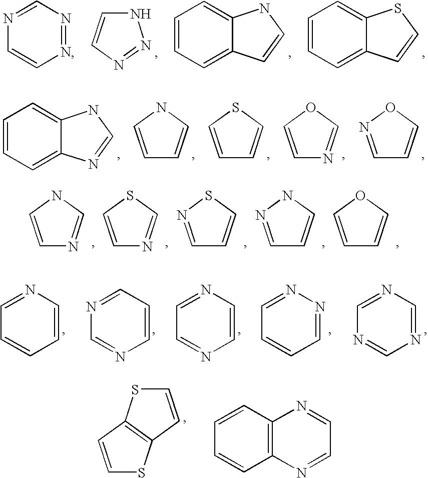

and the like. Depending on the structure, a heteroaryl group can be a monoradical or a diradical (i.e., a heteroarylene group).

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include: 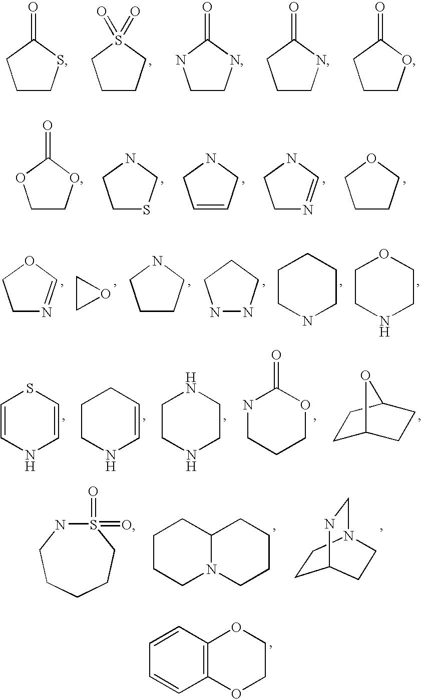
and the like. The term heteroalicyclic also includes all ring forms of the carbohydrates, including but not limited to the monosaccharides, the disaccharides and the oligosaccharides.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring can have additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms can be the same or different from one another. Heterocycles can be optionally substituted. Binding to a heterocycle can be at a heteroatom or via a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

The term “moiety” refers to a specific segment or functional group of a molecule. Chemical moieties are often recognized chemical entities embedded in or appended to a molecule.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, mercapto, alkylthio, arylthio, alkylsulfoxide, arylsulfoxide, alkylsulfone, arylsulfone, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. By way of example an optional substituents may be L_{S}R_{S}, wherein each L_{S} is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each R_{S} is independently selected from H, (substituted or unsubstituted lower alkyl), (substituted or unsubstituted lower cycloalkyl), heteroaryl, or heteroalkyl. The protecting groups that may form the protective derivatives of the above substituents are known to those of skill in the art and may be found in references such as Greene and Wuts, above.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutically acceptable salt” refers to a formulation of a compound that does not cause significant irritation to an organism to which it is administered and does not abrogate the biological activity and properties of the compound. Pharmaceutically acceptable salts may be obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts also may be obtained by reacting a compound described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art.

A “subject,” as referred to herein, can be any vertebrate (e.g., a mouse, rat, cat, guinea pig, hamster, rabbit, zebrafish, dog, non-human primate, or human) unless specified otherwise.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The compounds presented herein may possess one or more stereocenters and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds described herein, as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

Throughout the specification, groups and substituents thereof can be chosen by one skilled in the field to provide stable moieties and compounds.

Other features, objects, and advantages will be apparent from the description and from the claims.

### DESCRIPTION OF DRAWINGS

FIG. 1 is a representative plot of a dose range finding study in rats for peroral administration of a suspension of KDS-4103.

FIGS. 2 (A-B) are representative scatter plots depicting in rat the relationship between the level of FAAH inhibition in brain, and plasma levels of oleoylethanolamide (OEA) and palmitoylethanolamide (PEA), after oral administration of KDS-4103.

FIG. 3 is a representative scatter plot depicting in rat the relationship between OEA plasma levels and increase in AEA brain levels, after oral administration of KDS-4103.

FIG. 4 is a representative scatter plot depicting in rat the relationship between AEA brain levels and FAAH inhibition in the brain.

FIG. 5 is a representative plot depicting in rat the relationship between OEA plasma levels and oral dose of KDS-4103.

FIG. 6 is a representative plot depicting in rat the relationship between FAE plasma levels following escalating oral doses of KDS-4103.

FIG. 7 is a representative plot depicting in rats the time course of the mean OEA plasma concentration following a 50 mg/kg oral dose of KDS 4103.

FIG. 8 is a representative plot depicting in rats the time course of the mean OEA plasma concentration following a 275 mg/kg oral dose of KDS 4103.

FIG. 9 is a representative plot depicting in rats the time course of the mean OEA plasma concentration following a 1500 mg/kg oral dose of KDS 4103.

FIG. 10 is a representative plot depicting in monkey the time course (on day 1) of the mean OEA plasma concentration following different oral doses of KDS 4103.

FIG. 11 is a representative plot depicting in monkey the time course (on day 28) of the mean OEA plasma concentration following different oral doses of KDS 4103.

FIG. 12 is a representative plot depicting in mice the relationship between FAAH inhibition in brain and the oral dose of KDS-4103.

FIG. 13 is a representative plot depicting in mice the relationship between OEA plasma levels and FAAH inhibition in the brain following an oral dose of KDS-4103.

### DETAILED DESCRIPTION OF INVENTION

Physiological levels of FAAs are tightly regulated by the activity of fatty acid amide hydrolase (FAAH), a membrane-bound intracellular enzyme expressed in the nervous system. FAAH activity is particularly high in neocortex, hippocampus, and basal ganglia. Systemically administering FAAH inhibitors increases anandamide and cannabinoid receptor activity levels primarily within these regions, rather than ubiquitously as occurs following systemic anandamide administration. Indeed, FAAH inhibitors confer the same therapeutic effects as anandamide, but without the accompanying adverse side effects. In addition, reduced FAAH activity also leads to increased levels of non-cannabinergic FAAs (e.g., oleoylethanolamide). Some of these act via peroxisome proliferator-activated receptor α (PPAR-α) to modulate signaling pathways that underlie metabolic conditions such as insulin resistance, diabetes, hyperlipidemia, and obesity.

Effective dosing of FAAH inhibitors can be determined by assaying the ability of test compositions to increase systemic levels of one or more FAAs in a subject. To this end, systemic FAA levels can be assayed before and after administration of the test composition. FAAs that are generally present at relatively higher levels than anandamide physiologically are particularly useful, as they are easier to measure, yet highly correlated to changes in the level of FAAH activity in the brain, as well as to changes in anandamide levels. Suitable FAAs include oleolylethanolamide (OEA), palmitoylethanolamide (PEA), and stearoylethanolamide (SEA).

Alternatively, levels of various classes or subclasses of FAAs can be determined (e.g., saturated, polyunsaturated, and unsaturated fatty acid ethanolamides). Biological samples from which FAA levels can be assayed are, e.g., plasma, serum, blood, and cerebrospinal fluid, saliva, or urine.

FAA levels in a biological sample are assayed, e.g., by liquid chromatography tandem-mass spectrometry (LC-MS/MS). Increased assay reproducibility is achieved by spiking biological samples with a known amount of an isotopically labeled FAA, which serves as an internal standard for the FAA to be assayed. The level of the FAA can also be determined using spectrophotometric techniques (e.g., a fluorometric method). Alternatively, the level of the FAA can be determined using a biological assay. In some embodiments, the level of the FAA is determined using a combination of the aforementioned techniques. Any of the foregoing assays for FAA levels can be partly or fully automated for high throughput. Details of this and other FAA assays, as well as methods for analyzing changes in FAA levels are known in the art. See, e.g., Quistad et al. (2002), Toxicology and Applied Pharmacology 179: 57-63; Quistad et al. (2001), Toxicology and Applied Pharmacology 173, 48-55; Boger et al. (2000), Proc. Natl. Acad. Sci. U.S.A. 97, 5044-49; Cravatt et al. Proc. Natl. Acad. Sci. U.S.A. 98, 9371-9376 (2001); Ramarao et al. (2005), Anal Biochem. 343: 143-51. See also U.S. Pat. No. 6,096,784, U.S. patent application Ser. No. 10/681,858, PCT Publication WO 98/24396, and PCT Publication WO 04/033422.

Test compositions are administered at a range of doses spanning at least one log unit (e.g., about 0.5-about 5 mg/kg). In one embodiment, the range of administered doses results in no less than about 90 percent (e.g., any percent between about 90 and about 100) saturation of FAAH inhibition as reflected by a corresponding percent saturation in the amount of one or more FAA levels. For example, in administering a test composition to adult humans, dosages from about 10 to about 1000 mg, about 100 to about 500 mg or about 1 to about 100 mg may be needed. Doses of about 0.05 to about 100 mg, about 0.1 to about 100 mg, per day or up to four times a day may be used. In another embodiment the dosage is about 0.1 mg to about 70 mg per day. In choosing a regimen for patients, it may frequently be necessary to begin with a dosage of from about 2 to about 70 mg per day and when the condition is under control to reduce the dosage as low as from about 0.1 to about 10 mg per day. For example, in the treatment of adult humans, dosages from about 0.05 to about 100 mg, from about 0.1 to about 100 mg, per day may be used. The exact dosage will depend upon the mode of administration, on the level of FAAH inhibition required, the dosage form, the preference of the physician or veterinarian in charge, and the physiological characteristics of the subject. For example, the starting level of one or more FAAs can vary between individuals, and within individuals, e.g., according to a fasting state or disease state.

In some embodiments, baseline values of a systemic FAA level are determined in a subject before administering a test composition. After administration, FAA levels are assayed in biological samples obtained from the subject subsequent time points between (e.g., about 0.5 to about 25 hours post-administration). At least one additional time point is sufficient, but additional time points can also be used to further analyze the effect of the FAAH inhibitor. Systemic FAA levels may also be assayed at regular time intervals throughout the whole monitoring period, e.g., at 1, 2, or 3 hour intervals. FAA levels for each time point can then be normalized relative to baseline values. Changes in systemic FAA levels can be analyzed in different ways, e.g., as changes in Cₘₐₓ, Tₘₐₓ, or AUC, or as overall exposure) according to preferred criteria. For example, Tₘₐₓ analysis is particularly important when the FAAH inhibitor is to be used for the treatment of acute conditions, i.e., conditions that require rapid onset of FAAH inhibition.

A dose of a test composition that induces a statistically significant increase of at least about 50% in one or more FAA levels relative to baseline values is considered to be effective for inhibiting FAAH activity in vivo. In further embodiments, a statistically significant increase of at least about 70% in one or more FAA levels relative to baseline values is considered to be effective for inhibiting FAAH activity in vivo. a statistically significant increase of at least about 80% in one or more FAA levels relative to baseline values is considered to be effective for inhibiting FAAH activity in vivo.

In another embodiment, FAA levels determined after administering a test composition can be compared to one or more pre-determined values, rather than, or in addition to, the subject's own baseline FAA levels. For example, comparisons can be made to a systemic FAA level corresponding to the mean, median, or mode of FAA levels in a population of subjects administered a vehicle (or not administered any composition).

A subject in the method described herein can be healthy or can suffer from a condition ameliorated by inhibiting FAAH activity. Such conditions include pain, e.g., nociceptive pain, neuropathic pain, inflammatory pain, non-inflammatory pain, painful hemorrhagic cystitis, pain associated with the herpes virus, pain associated with diabetes, peripheral neuropathic pain, peri-operative pain, cancer pain, pain and spasticity associated with multiple sclerosis, central pain, deafferentiation pain, chronic nociceptive pain, stimulus of nociceptive receptors, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain; psychiatric disorders, e.g., anxiety, stress, obsessive-compulsive disorder, depression, panic disorder, psychosis, addiction, alcoholism, attention deficit hyperactivity, agoraphobia, schizophrenia, or social phobia; neurological disorders, e.g., epilepsy, stroke, insomnia, diskinesia, peripheral neuropathic pain, chronic nociceptive pain, phantom pain, deafferentation pain, inflammatory pain, joint pain, wound pain, post-surgical pain, or recurrent headache pain, appetite disorders, or motor activity disorders; neurodegenerative diseases, e.g., Alzheimer's disease, Parkinson's disease, or Huntington's disease; inflammatory conditions, e.g., arthritis, chronic obstructive pulmonary disease, tendonitis, or hepatitis; and metabolic syndromes, e.g., insulin resistance syndrome, diabetes, hyperlipidemia, obesity, fatty liver disease, arteriosclerosis, or atherosclerosis. Symptoms and appropriate diagnostic tests for each of the just-mentioned conditions are known in the art. See, e.g., “Harrison's Principles of Internal Medicine,” ©, 16^{th} ed., 2004, The McGraw-Hill Companies, Inc.

The FAA level obtained following administration of the FAAH inhibitor can be used by a physician (or any person responsible for determining the dose of a FAAH inhibitor provided to a subject/patient) as one indication that the current dose level of the FAAH inhibitor is sufficient for subsequent doses or needs to be adjusted. For example, if the FAA level is above a pre-determined value, then the physician can use such information that the current dose of the FAAH inhibitor is sufficient for subsequent doses. Of course, if the health of the patient has not improved (e.g., the pain experienced by the patient has not been sufficiently alleviated), then the physician can use his/her judgment to raise the subsequent dose. As another example, if the FAA level is below a pre-determined value, then the physician can use such information that the current dose of the FAAH inhibitor is insufficient for subsequent doses, and the subsequent doses of the FAAH inhibitor can be raised. Of course, if the health of the patient has improved even though the FAA level is below the predetermined level, then the physician can use his/her judgment to not raise the subsequent dose. In other words, the FAA levels are one factor that a physician can consider in determining the appropriateness of a dose of FAAH inhibitor.

It is to be understood that where the subject is a non-human, it can exhibit a condition that corresponds to or closely resembles the equivalent human disease state. Indeed, a number of animal models of human disease are very useful for establishing therapeutically effective doses of FAAH inhibitors. More specifically, relief of symptoms for a particular condition can be correlated to a specific level of FAAH inhibition observed for a given dose of a FAAH inhibitor vis-a-vis the measured level of one or more FAAs.

For example, dosing of FAAH inhibitors for treating pain can be evaluated using the “PPQ test,” a visceral pain model described in Pearl et al. (1968) J. Pharmacol Exp. Ther., 160:217-230. Alternatively, analgesic properties of FAAH inhibitors can be determined in the mouse hot-plate test or the mouse formalin test and the nociceptive reactions to thermal or chemical tissue damage measured. See, e.g., U.S. Pat. No. 6,326,156 which teaches methods of screening for antinociceptive activity, and Cravatt et al. (2001) Proc. Natl. Acad. Sci. U.S.A., 98:9371-9376.

Levels of FAAH inhibition effective for treating anxiety disorders can also be assessed in animal models. Two pharmacologically validated animal models of anxiety are the elevated zero maze test, and the isolation-induced ultrasonic emission test. The zero maze consists of an elevated annular platform with two open and two closed quadrants and is based on the conflict between an animal's instinct to explore its environment and its fear of open spaces, where it may be attacked by predators. See, e.g., Bickerdike et al. (1994), Eur. J. Pharmacol., 271, 403-411; and Shepherd et al. (1994), Psychopharmacology, 116, 56-64 (1994). Clinically used anxiolytic drugs, such as benzodiazepines, increase the proportion of time spent in, and the number of entries made into, the open compartments.

A second test for quantifying a level of anxiety is the ultrasonic vocalization emission model, which measures the number of stress-induced vocalizations emitted by rat pups removed from their nest. See Insel, T. R. et al. (1986), Pharmacol. Biochem. Behav., 24, 1263-1267; Miczek et al. (1995), Psychopharmacology, 121, 38-56; and Winslow et al., Biol. Psychiatry, 15, 745-757 (1991). See also U.S. Pat. No. 6,326,156.

Other animal models of anxiety evaluate behavior in a so-called “conflict” situation, i.e., where a behavioral response is simultaneously under the influence of two opposing motivational states such as approach and avoidance tendencies. Probably the best known model is the conditioned punishment conflict paradigm in which animals are trained to voluntarily exhibit a certain response (e.g., pressing a lever) in order to receive a reward (e.g. food for a hungry animal). Once the animals exhibit a constant rate of lever-press responding, short periods are introduced (usually signaled by visual or acoustic signals) during which lever pressing is simultaneously rewarded by food and punished by mild electrical foot shock. Animals exhibit a markedly reduced response rate during these conflict periods, which are also characterized by various overt signs of emotionality. The characteristic effect of benzodiazepine receptor agonists, for example the anxiolytic diazepam, is the disinhibition of punished behavior (resulting in an increase in the punished response rate) at doses that fail to disrupt unpunished responses. Further, these drugs produce an anxiolytic-like effect in the absence of actual punishment, i.e., when the rate of lever pressing is reduced by conditioned fear of punishment. The conflict task does not require conditioned behavioral responses: naive thirsty animals can be offered the opportunity to drink, with drinking punished by contact with an electrified spout. Such punishment-suppressed drinking is disinhibited in a dose-dependent manner by benzodiazepine receptor agonists (e.g., diazepam). Exploratory activity can likewise be decreased by contingent punishment and released by treatment with known anxiolytics. Conflict models without punishment are based on the presence of the natural opposing motivational states, on the one hand the tendency to explore and, on the other hand, fear of a novel environment (e.g., dark-light chamber task, elevated plus maze, consumption of unfamiliar food or normal food in an unfamiliar environment and social interaction between animals unfamiliar with each other).

Effective doses of FAAH inhibitors can be quantified in the “chronic mild stress induced anhedonia” model, which is based on the observation that chronic mild stress causes a gradual decrease in sensitivity to rewards, for example consumption of sucrose, and that this decrease is dose-dependent and reversed by chronic treatment with antidepressants. See Willner (1997), Psychopharmacology, 134, 319-329. Another useful test for quantifying the antidepressant activity of FAAH inhibitors is the forced swimming test. See Porsolt et al. (1977), Nature 266, 730-732. In this test, animals are administered a FAAH inhibitor test composition preferably by the oral route 30 or 60 minutes before the test. Subsequently, the animals are placed in a crystallizing dish filled with water and the time during which they remain immobile is clocked. The immobility time is then compared with that of the control group treated with distilled water. Imipramine 25 mg/kg can be used as a positive control. Antidepressant compounds decrease the immobility time of the mice thus immersed. Another test for antidepressant activity is the caudal suspension test on the mouse Psychopharmacology, 85, 367-370, 1985). In this test, animals are preferably treated with the compound by the by the oral or intraperitoneal route 30 minutes to 6 hours before the test or. The animals are then suspended by the tail and their immobility time is automatically recorded by a computer system. Immobility times are then compared with those of a control group treated with vehicle. Imipramine 25 mg/kg can be used as the positive control. Antidepressant compounds decrease the immobility time of the mice. Antidepressant effects of FAAH inhibitors can be tested in the DRL-72 TEST. This test, carried out according to the protocol of Andrews et al. (1994), Drug Development Research 32, 58-66, gives an indication of antidepressant-like activity. See U.S. Pat. Nos. 6,403,573 and 5,952,315.

Effective levels of FAAH inhibition can also be determined in animal models of metabolic disorders, e.g., those disclosed in U.S. Pat. No. 6,946,491.

A number of transgenic mouse models of neurodegenerative disorders (e.g., Alzheimer's disease, and amylotrophic lateral sclerosis) have been established. See, e.g., Spires et al. (2005), NeuroRx., 2(3):447-64 and Wong et al. (2002), Nat. Neurosci., 5(7):633-639. Such transgenic animal models spontaneously develop a neurodegenerative disorder that is manifested behaviorally by impaired learning, memory, or locomotion. Such animal models are suitable for determining effective doses of FAAH inhibitors. Cognitive abilities, as well as motor functions in non-human animals suffering from a neurodegenerative disorder can be assessed using a number of behavioral tasks. Well-established sensitive learning and memory assays include the Morris Water Maze (MWM), context-dependent fear conditioning, cued-fear conditioning, and context-dependent discrimination. See, e.g., Anger (1991), Neurotoxicology, 12(3):403-413. Examples of motor behavior/function assays, include the rotorod test, treadmill running, and general assessment of locomotion.

Once effective doses of FAAH inhibitors for a particular disease condition have been determined, e.g., in one of the foregoing assays, therapeutic efficacy can be optimized during a course of treatment. The subject can undergo a diagnostic evaluation to determine the severity of symptoms vis-a-vis systemic FAA levels. The amount of FAAH inhibitor administered to the subject is then increased or decreased as needed so as to maintain a level of FAAH inhibition optimal for treating the condition.

Generally, a FAAH inhibitor used in the methods described herein is identified as an inhibitor of FAAH in vitro. Preferred in vitro assays detect binding of an inhibitor compound to FAAH or the release of a reaction product (e.g., fatty acid amide or ethanolamine) produced by the hydrolysis of a substrate such as anandamide or OEA. The substrate may be labeled to facilitate detection of the released reaction products. High throughput assays for the presence, absence, or quantification of particular reaction products are well known to those of ordinary skill in the art. In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. Automated systems thereby allow the identification of a large number of in vitro FAAH inhibitors without undue effort.

Examples of FAAH Inhibitors

In the following description of FAAH inhibitors suitable for use in the methods described herein, definitions of standard chemistry terms may be found in reference works (if not otherwise defined herein), including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

The FAAH inhibitor compositions used in the methods described herein can come from a variety of sources including both natural (e.g., plant extracts) and synthetic. Candidate FAAH inhibitor composition can be isolated from a combinatorial library, i.e., a collection of diverse chemical compounds generated by either chemical synthesis or biological synthesis by combining a number of chemical “building blocks.” For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. Indeed, theoretically, the systematic, combinatorial mixing of 100 interchangeable chemical building blocks results in the synthesis of 100 million tetrameric compounds or 10 billion pentameric compounds. See Gallop et al. (1994), J. Med. Chem. 37(9), 1233. Preparation and screening of combinatorial chemical libraries are well known in the art. Combinatorial chemical libraries include, but are not limited to: diversomers such as hydantoins, benzodiazepines, and dipeptides, as described in, e.g., Hobbs et al. (1993), Proc. Natl. Acad. Sci. U.S.A. 90, 6909; analogous organic syntheses of small compound libraries, as described in Chen et al (1994), J. Amer. Chem. Soc., 116: 2661; Oligocarbamates, as described in Cho, et al. (1993), Science 261, 1303; peptidyl phosphonates, as described in Campbell et al. (1994), J. Org. Chem., 59: 658; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514).

Devices for the preparation of combinatorial libraries are commercially available (see, e.g., 357 MPS, 390 MPS from Advanced Chem Tech, Louisville, Ky.; Symphony from Rainin, Woburn, Mass.; 433A from Applied Biosystems, Foster City, Calif.; and 9050 Plus from Millipore, Bedford, Mass.). A number of well known robotic systems have also been developed for solution phase chemistries. These systems include automated workstations like the automated synthesis apparatus developed by Takeda Chemical Industries, LTD (Osaka, Japan), and many robotic systems utilizing robotic arms (Zymate II, Zymark Corporation, Hopkinton, Mass.; Orca, Hewlett Packard, Palo Alto, Calif.) which mimic the manual synthetic operations performed by a chemist. Any of the above devices can be used to characterize functional analogues and to evolve variants of the FAAH inhibitors used in the methods disclosed herein. In addition, numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.).

A composition used in the methods described herein can inhibit FAAH activity, in vitro, with an IC₅₀ of less than about 10 μM (e.g., about 1 μM, about 0.5 μM, or about 0.01 μM).

In one embodiment, the fatty acid amide hydrolase inhibitor is an alkylcarbamic acid aryl ester of Formula (I): 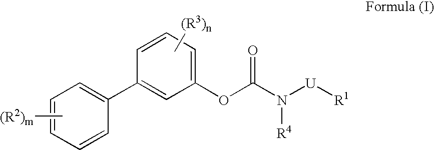
wherein:
R¹ is selected from among C₁-C₈ alkyl, C₁-C₄alkyl-(C₃-C₈cycloalkyl), and C₃-C₈ cycloalkyl (e.g., cyclohexyl); R⁴ is H or alkyl;
R² and R³ are each independently selected from among H, C₁-C₄ alkyl, C₂-C₄ alkenyl, C₂-C₄ alkynyl, C₃-C₆ cycloalkyl, C₁-C₄alkyl-(C₃-C₆cycloalkyl), aryl, substituted aryl, arylalkyl, —C(O)R^{A}, hydroxy-(C₁-C₆ alkyl), amino-(C₁-C₆ alkyl), —CH₂—NR^{A}R^{B}, —O—(C₁-C₄), aryloxy, halo, C₁-C₆-haloalkyl, cyano, hydroxy, nitro, amino, —C(O)NR^{A}R^{B}, —ONR^{A}R^{B}, —O—C(O)NR^{A}R^{B}, —SO₂NR^{A}R^{B};
R^{A} and R^{B} are each independently selected from among hydrogen, C₁-C₆ alkyl, and C₃-C₆ cycloalkyl;
U is a bond or CH₂;

m and n are each independently 0-3; and

pharmaceutically acceptable salts, pharmaceutically acceptable N-oxides, pharmaceutically active metabolites, pharmaceutically acceptable prodrugs, or pharmaceutically acceptable solvates thereof.

In another embodiment, the alkylcarbamic acid aryl ester has the structure of compound KDS-4103:

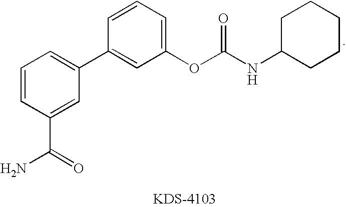

In a further embodiment, the test composition is an alkylcarbamic acid aryl ester of Formula (II): 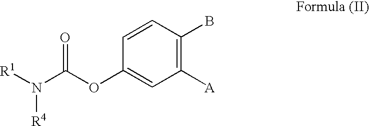
wherein:
R¹ is selected from among C₁-C₈ alkyl, C₁-C₄alkyl-(C₃-C₈cycloalkyl), and C₃-C₈ cycloalkyl (e.g., cyclohexyl); R⁴ is H or alkyl;
A and B are selected from:
   (i) one of A or B is C(O)-alkyl, and the other is H, alkyl, heteroalkyl; A and B can combine into a non-aromatic cyclic group; A and B can be substituted;
   (ii) A and B together form an optionally substituted heteroaromatic group; A and/or B are N, S, or O; or
   (iii) one of A or B is L-X-G; the other is H, alkyl; L is optionally substituted alkyl or heteroalkyl; X is a bond, O, —C(═O), —CR₉(OR₉), S, —S(═O), —S(═O)₂, —NR₉, —NR₉C(O), —C(O)NR₉, —S(═O)₂NR₉—, —NR₉S(═O)₂, —OC(O)NR₉—, —NR₉C(O)O—, —NR₉C(O)NR₉—, —NR₉C(═NR₁₀)NR₉—, —NR₉C(═NR₁₀)—, —C(═NR₁₀)NR₉—, —OC(═NR₁₀)—, or —C(═NR₁₀)O—; G is H, tetrazolyl, —NHS(═O)₂R₈, S(═O)₂N(R₉)₂, —OR₉, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(O)NR₉C(═NR₁₀)N(R₉)₂, —C(O)NR₉C(═CR₁₀)N(R₉)₂, —CO₂R₉, —C(O)R₉, —CON(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(O)O—, —NHC(O)NH—, —NHC(O)O, —O(O)CNH—, —NHC(O), —C(O)NH, —C(O)O, or —OC(O); each R₈ is independently selected from substituted or unsubstituted lower alkyl; each R₉ is independently selected from H, substituted or unsubstituted lower alkyl; and each R₁₀ is independently selected from H, —S(═O)₂R₈, —S(═O)₂NH₂—C(O)R₈, —CN, or —NO₂; and

pharmaceutically acceptable salts, pharmaceutically acceptable N-oxides, pharmaceutically active metabolites, pharmaceutically acceptable prodrugs, or pharmaceutically acceptable solvates thereof.

In yet a further embodiment, is a method described herein, wherein the fatty acid amide hydrolase inhibitor can be an alkylcarbamic acid aryl ester of Formula (II):

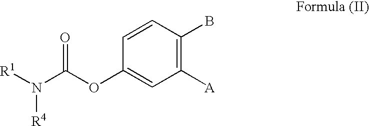

wherein:
R¹ is selected from among C₁-C₈ alkyl, C₁-C₄alkyl-(C₃-C₈cycloalkyl), and C₃-C₈ cycloalkyl (e.g., cyclohexyl); R⁴ is H or alkyl;
one of A or B is L-X-G; the other is H, alkyl;
L is optionally substituted alkyl;
X is a bond;
G is —CO₂R₉;
R₉ is H; and

pharmaceutically acceptable salts, pharmaceutically acceptable N-oxides, pharmaceutically active metabolites, pharmaceutically acceptable prodrugs, or pharmaceutically acceptable solvates thereof.

In another embodiment, is a method described herein, wherein the test composition is an alkylcarbamic acid aryl ester of Formula (II): 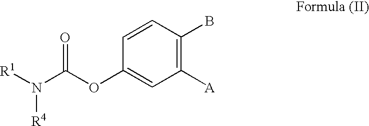
wherein:
R¹ is selected from among C₁-C₈ alkyl, C₁-C₄alkyl-(C₃-C₈cycloalkyl), and C₃-C₈ cycloalkyl (e.g., cyclohexyl); R⁴ is H or alkyl;
   A and B together form an optionally substituted heteroaromatic group; A and/or B are N, S, or O; and

pharmaceutically acceptable salts, pharmaceutically acceptable N-oxides, pharmaceutically active metabolites, pharmaceutically acceptable prodrugs, or pharmaceutically acceptable solvates thereof.

In another embodiment, A and B together form an optionally substituted heteroaromatic group wherein A and B is N or S. In a further embodiment, the heteroaromatic group wherein A and B is N or S, is CH₃ substituted. In another embodiment, A and B together form an optionally substituted heteroaromatic group wherein A and B is N or O. In yet a further embodiment, the heteroaromatic group wherein A and B is N or O is CH₃ substituted. In a further embodiment, A and B form a heteroaromatic group wherein A and B are N and the heteroaromatic group is a tetrazolyl group.

In one embodiment, is a method described herein, wherein the fatty acid amide hydrolase inhibitor can be an alkylcarbamic acid aryl ester of Formula (IIa): 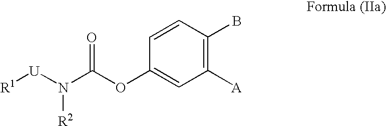
wherein:
R¹ is substituted or unsubstituted C₃-C₉ alkyl (including linear, branched, cyclic alkyl groups and combinations thereof);
R² is H or an optionally substituted alkyl;
U is a bond or CH₂;
one of A or B is (CH₂)_{q}C(O)-alkyl, (CH₂)_{q}C(O)—N(R²)₂ and the other is H, alkyl, or heteroalkyl, q is 0, 1, 2, 3, or 4;
or A and B together form an optionally substituted C(O)—(CH₂)_{q}— moiety, wherein q is 1, 2, 3 or 4;
or A and B together form an optionally substituted heteroaromatic group comprising at least one N, NR², S, or O group;
or A and B together form an optionally substituted non-aromatic or aromatic carbocycle group;
or A and B together form an optionally substituted oxo-substituted heterocycle;
or A and B are each independently selected from among H, an optionally substituted alkyl, an optionally substituted heteroalkyl, an optionally substituted heterocyclic group, an optionally substituted aryl group, an optionally substituted heteroaryl group, an optionally substituted ketoalkyl, an optionally substituted amide, and an optionally substituted ketoheteroalkyl;
one of A or B is -L-G and the other is selected from among H and an optionally substituted C₁-C₆ alkyl; or
L is a bond, or an optionally substituted group selected from among C₁-C₆ alkylene, C₁-C₆ heteroalkylene, C₁-C₆ ketoalkylene, a monosaccharide, a disaccharide, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O—(CH₂—CH₂—O)_{q}—H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H₃, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —(OP(═O)OH)ₙOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁹, —CN, and —NO₂;
n is 1, 2, 3, or 4; x is 1, 2, or 3; y is 0, 1, or 2; and
pharmaceutically acceptable salts, pharmaceutically acceptable N-oxides, pharmaceutically active metabolites, pharmaceutically acceptable prodrugs, or pharmaceutically acceptable solvates thereof.

In one embodiment, is a method described herein, wherein the fatty acid amide hydrolase inhibitor can be an alkylcarbamic acid aryl ester of Formula (IIa) is:

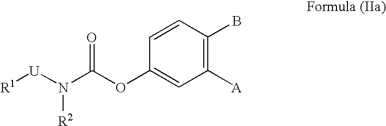

wherein:

R² is H or an optionally substituted alkyl;

U is a bond or CH₂;

R¹ is a substituted or unsubstituted C₃-C₉ cyclic alkyl;

A and B are individually R²¹ and (V—R²⁴);

R²¹ is H, a carboxylic acid bioisostere, or a C₃-C₇ heterocycle;

V is a bond, CH₂, NH, or NR²⁵;

R²⁴ is H, —(CO)R²⁵, —CO₂H, a carboxylic acid bioisostere, —(CO)NH₂, —(CO)NHR²⁵, —NH(CO)R²⁵, —NR²⁵ (CO)R²⁷; or
R²¹ and R²⁴ together form an optionally substituted C₅-C₆ heterocycle;
R²⁵ and R²⁷ are independently selected from H or an alkyl group; and

pharmaceutically acceptable salts, pharmaceutically acceptable N-oxides, pharmaceutically active metabolites, pharmaceutically acceptable prodrugs, or pharmaceutically acceptable solvates thereof.

In a further embodiment, is a method described herein, wherein the fatty acid amide hydrolase inhibitor can be an alkylcarbamic acid aryl ester of Formula (VII):

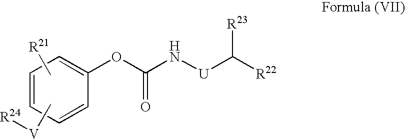

wherein:
U is a bond or CH₂;
V is a bond, CH₂, NH, or NR²⁵;
R²¹ is H, a carboxylic acid bioisostere, or a C₃-C₇ heterocycle;
R²² and R²³ are individually H, C₁-C₈ alkyl, (C₃-C₇ cycloalkyl), (C₁-C₄ alkyl(C₃-C₇ cycloalkyl)); or
R²² and R²³ together form a 3-, 4-, 5-, 6-, or 7-membered cycloalkyl or an oxygen containing heterocycloalkyl group;
R²⁴ is H, —(CO)R²⁵, —CO₂H, a carboxylic acid bioisostere, —(CO)NH₂, —(CO)NHR²⁵, NH(CO)R²⁵, —NR(CO)R²⁷; or
R²¹ and R²⁴ together form an optionally substituted C₅-C₆ heterocycle;
R²⁵ and R²⁷ are independently selected from H or an alkyl group; and
pharmaceutically acceptable salts, pharmaceutically acceptable N-oxides, pharmaceutically active metabolites, pharmaceutically acceptable pro drugs, or pharmaceutically acceptable solvates thereof.

In another embodiment, is a method described herein, wherein the fatty acid amide hydrolase inhibitor can be an alkylcarbamic acid aryl ester of Formula (VIII) comprising:

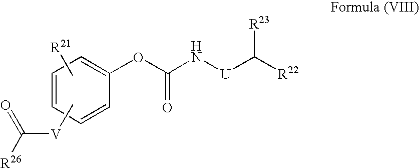

wherein:
U is bond or CH₂;
V is a bond, CH₂, NH, or NR
R²¹ is H;
R²² and R²³ are individually H, C₁-C₈ alkyl, (C₃-C₇ cycloalkyl), (C₁-C₄ alkyl(C₃-C₇ cycloalkyl)); or
R²² and R²³ together form a 3-, 4-, 5-, 6-, or 7-membered cycloalkyl or an oxygen containing heterocycloalkyl group;
R²⁵ is H or an alkyl group;
R²⁶ is R²⁵NH₂, —NR²⁵, or OH; and
pharmaceutically acceptable salts, pharmaceutically acceptable N-oxides, pharmaceutically active metabolites, pharmaceutically acceptable pro drugs, or pharmaceutically acceptable solvates thereof.

In yet another embodiment, is a method described herein, wherein the fatty acid amide hydrolase inhibitor can be an alkylcarbamic acid aryl ester of Formula (VIII) comprising:

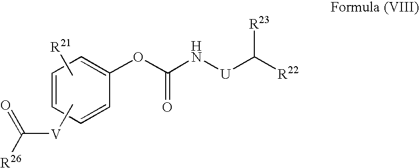

wherein:
U is a bond or CH₂;
V is a bond, CH₂, NH, or NR
R²¹ is H;
R²² and R²³ together form a 6-membered cycloalkyl group;
R²⁵ is H or an alkyl group;
R²⁶ is 5, NH₂, —NR²⁵, or OH; and
pharmaceutically acceptable salts, pharmaceutically acceptable N-oxides, pharmaceutically active metabolites, pharmaceutically acceptable pro drugs, or pharmaceutically acceptable solvates thereof.

In one embodiment, is a method described herein, wherein the fatty acid amide hydrolase inhibitor can be an alkylcarbamic acid aryl ester of Formula (IX) comprising:

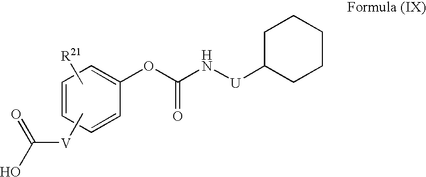

wherein:
U and V are individually a bond or CH₂;
R²¹ is H; and
pharmaceutically acceptable salts, pharmaceutically acceptable N-oxides, pharmaceutically active metabolites, pharmaceutically acceptable pro drugs, or pharmaceutically acceptable solvates thereof.

In one embodiment is a method described herein, wherein the compound of Formula (IX) is selected from the group consisting of:

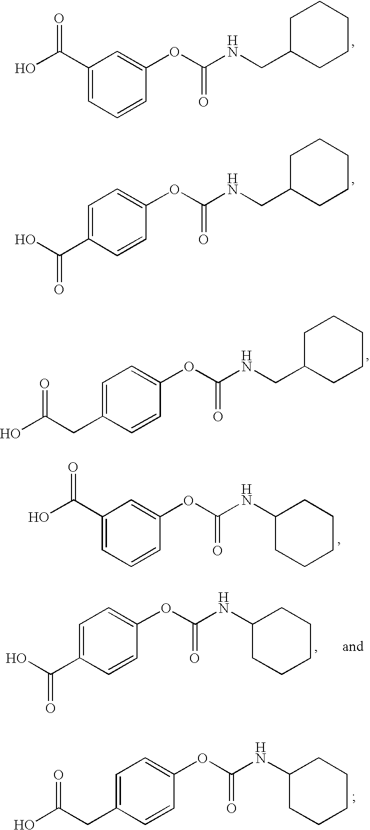

and pharmaceutically acceptable salts, pharmaceutically acceptable N-oxides, pharmaceutically active metabolites, pharmaceutically acceptable prodrugs, or pharmaceutically acceptable solvates thereof.

In one embodiment, is a method described herein, wherein the fatty acid amide hydrolase inhibitor can be an alkylcarbamic acid aryl ester of Formula (VIII) comprising:

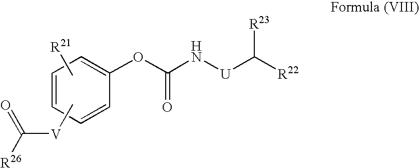

wherein:
U is a bond or CH₂;
V is NH or NR²⁵;
R²¹ is H;
R²² and R²³ together form a 6-membered cycloalkyl group;
R²⁵ and R²⁶ are individually an alkyl group; and
pharmaceutically acceptable salts, pharmaceutically acceptable N-oxides, pharmaceutically active metabolites, pharmaceutically acceptable pro drugs, or pharmaceutically acceptable solvates thereof.

In another embodiment is a method described herein, wherein the compound of Formula (VIII) is selected from the group consisting of:

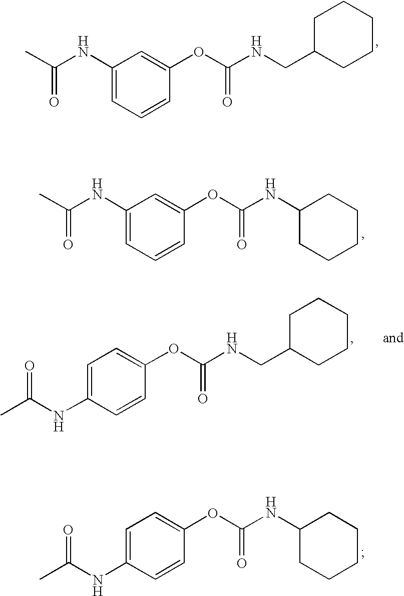

and pharmaceutically acceptable salts, pharmaceutically acceptable N-oxides, pharmaceutically active metabolites, pharmaceutically acceptable prodrugs, or pharmaceutically acceptable solvates thereof.

In one embodiment, is a method described herein, wherein the fatty acid amide hydrolase inhibitor can be an alkylcarbamic acid aryl ester of Formula (VIII) comprising:

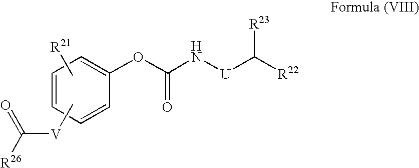

wherein:
U and V are individually a bond or CH₂;
R²¹ is H;
R²² and R²³ together form a 6-membered cycloalkyl group;
R²⁵ is an alkyl group;
R²⁶ is NH₂ or NHR²⁵; and
pharmaceutically acceptable salts, pharmaceutically acceptable N-oxides, pharmaceutically active metabolites, pharmaceutically acceptable pro drugs, or pharmaceutically acceptable solvates thereof.

In another embodiment is a method described herein, wherein the compound of Formula (VIII) is:

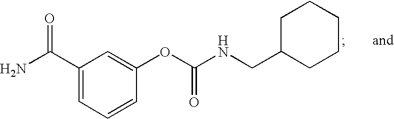

a pharmaceutically acceptable salts, pharmaceutically acceptable N-oxides, pharmaceutically active metabolites, pharmaceutically acceptable prodrugs, or pharmaceutically acceptable solvates thereof.

In a further embodiment, is a method described herein, wherein the compound of Formula (VIII) is:

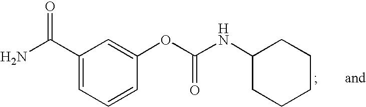

pharmaceutically acceptable salts, pharmaceutically acceptable N-oxides, pharmaceutically active metabolites, pharmaceutically acceptable prodrugs, or pharmaceutically acceptable solvates thereof.

In a further embodiment, is a method described herein, wherein the fatty acid amide hydrolase inhibitor can be an alkylcarbamic acid aryl ester of Formula (VII) comprising:

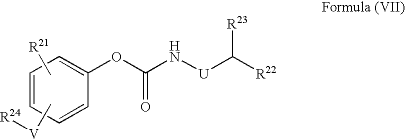

wherein:
U is a bond or CH₂;
V is a bond;
R²² and R²³ are individually H, C₁-C₈ alkyl, (C₃-C₇ cycloalkyl), (C₁-C₄ alkyl(C₃-C₇ cycloalkyl)); or
R²² and R²³ together form a 3-, 4-, 5-, 6-, or 7-membered cycloalkyl or an oxygen containing heterocycloalkyl group;
R²¹ and R²⁴ together form an optionally substituted C₅-C₆ heteroaryl; and
pharmaceutically acceptable salts, pharmaceutically acceptable N-oxides, pharmaceutically active metabolites, pharmaceutically acceptable pro drugs, or pharmaceutically acceptable solvates thereof.

In one embodiment is a method described herein, wherein a compound of Formula (X) comprises:

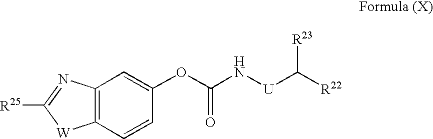

wherein:
U is a bond or CH₂;
R²² and R²³ together form a 6-membered cycloalkyl group;
W is O or S;
R²⁵ is H or an alkyl group; and
pharmaceutically acceptable salts, pharmaceutically acceptable N-oxides, pharmaceutically active metabolites, pharmaceutically acceptable pro drugs, or pharmaceutically acceptable solvates thereof.

In another embodiment is a method described herein, wherein the compound of Formula (XI) comprising:

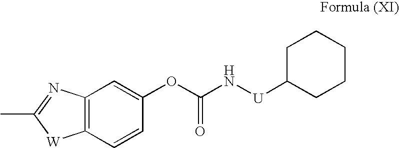

wherein:
U is a bond or CH₂;
W is O or S; and
pharmaceutically acceptable salts, pharmaceutically acceptable N-oxides, pharmaceutically active metabolites, pharmaceutically acceptable pro drugs, or pharmaceutically acceptable solvates thereof.

In another embodiment is a compound of Formula (XI) selected from the group consisting of:

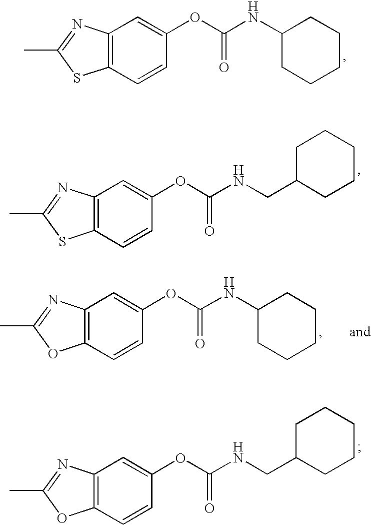

and pharmaceutically acceptable salts, pharmaceutically acceptable N-oxides, pharmaceutically active metabolites, pharmaceutically acceptable prodrugs, or pharmaceutically acceptable solvates thereof.

In one embodiment, the fatty acid amide hydrolase inhibitor can be an alkylcarbamic acid aryl ester of Formula (VII) comprising:

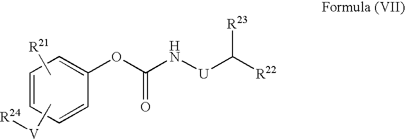

wherein:
U is a bond or CH₂;
V is a bond;
R²¹ is a C₃-C₇ heterocycle or a carboxylic acid bioisostere;
R²² and R²³ are individually H, C₁-C₈ alkyl, (C₃-C₇ cycloalkyl), (C₁-C₄ alkyl(C₃-C₇ cycloalkyl)); or
R²² and R²³ together form a 3-, 4-, 5-, 6-, or 7-membered cycloalkyl or an oxygen containing heterocycloalkyl group;
R²⁴ is H; and
pharmaceutically acceptable salts, pharmaceutically acceptable N-oxides, pharmaceutically active metabolites, pharmaceutically acceptable pro drugs, or pharmaceutically acceptable solvates thereof.

In one embodiment, the fatty acid amide hydrolase inhibitor can be an alkylcarbamic acid aryl ester of Formula (XII) comprising:

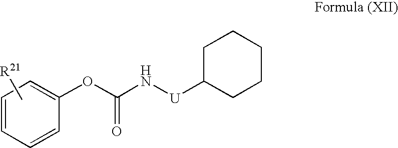

wherein:
U is a bond or CH₂;
R²¹ is a tetrazolyl group; and
pharmaceutically acceptable salts, pharmaceutically acceptable N-oxides, pharmaceutically active metabolites, pharmaceutically acceptable pro drugs, or pharmaceutically acceptable solvates thereof.

In yet a further embodiment is a method described herein, wherein the compound of Formula (XII) selected from the group consisting of: 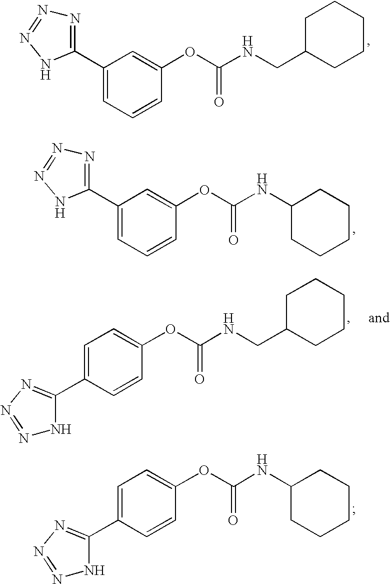
and pharmaceutically acceptable salts, pharmaceutically acceptable N-oxides, pharmaceutically active metabolites, pharmaceutically acceptable prodrugs, or pharmaceutically acceptable solvates thereof.

In one aspect is a method for determining an effective dose of a composition for increasing endogenous levels of anandamide in a subject comprising:

determining a level of at least one fatty acid amide other than anandamide in a biological sample obtained from a subject at a first time point;

determining a level of the at least one fatty acid amide in a biological sample obtained from the subject at a second time point, wherein prior to the second time point the subject has been administered a dose of a fatty acid amide hydrolase inhibitor, and indicating that the dose is effective when the level of the at least one fatty acid amide is determined to be greater in the biological sample obtained at the second time point than at the first time point.

In one embodiment, the biological samples obtained at the first and second time points are plasma, whole blood, serum, saliva, or cerebrospinal fluid. In another embodiment, the sample obtained at the first and second time points is plasma. In a further embodiment, the sample obtained at the first and second time points is whole blood. In another embodiment, the sample obtained at the first and second time points is saliva.

In one embodiment, the fatty acid amide is oleoylethanolamide. In another embodiment, the fatty acid amide is palmitoylethanolamide. In a further embodiment, the fatty acid amide is stearoylethanolamide. In yet a further embodiment, the fatty acid hydrolase inhibitor is administered orally.

In one embodiment, the dose is concluded to be effective when the level of the at least one fatty acid amide at the second time point is at least about 50% greater than at the first time point. In another embodiment, the dose is concluded to be effective when the level of the at least one fatty acid amide at the second time point is at least about 90% of its saturation value. In yet another embodiment, the dose is concluded to be effective when the level of the at least one fatty acid amide at the second time point is saturated. In a further embodiment, the subject is a human. In yet a further embodiment, the subject is a non-human primate. In one embodiment, the subject is suffering from a psychiatric, neurological, neurodegenerative, painful, or metabolic disorder.

In one embodiment the subject is suffering from a pain disorder selected from the group consisting of nociceptive pain, neuropathic pain, inflammatory pain, non-inflammatory pain, painful hemorrhagic cystitis, pain associated with the herpes virus, pain associated with diabetes, peripheral neuropathic pain, peri-operative pain, cancer pain, pain and spasticity associated with multiple sclerosis, central pain, deafferentiation pain, chronic nociceptive pain, stimulus of nociceptive receptors, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

In another embodiment, the subject is suffering from a metabolic disorder.

In one embodiment, the method further comprises performing a diagnostic evaluation of the subject before and after administering the test composition.

In one embodiment, is a method for determining an effective dose of a composition for increasing endogenous levels of anandamide in a subject, wherein the fatty acid amide hydrolase inhibitor is an alkylcarbamic acid aryl ester of Formula (I):

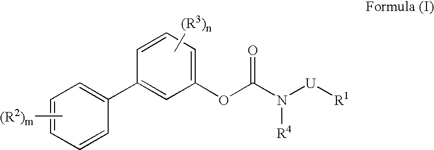

wherein:
R¹ is selected from among C₁-C₈ alkyl, C₁-C₄alkyl-(C₃-C₈cycloalkyl), and C₃-C₈ cycloalkyl (e.g., cyclohexyl); R⁴ is H or alkyl;
R² and R³ are each independently selected from among H, C₁-C₄ alkyl, C₂-C₄ alkenyl, C₂-C₄ alkynyl, C₃-C₆ cycloalkyl, C₁-C₄alkyl-(C₃-C₆cycloalkyl), aryl, substituted aryl, arylalkyl, —C(O)R^{A}, hydroxy-(C₁-C₆ alkyl), amino-(C₁-C₆ alkyl), —CH₂—NR^{A}R^{B}, —O—(C₁-C₄), aryloxy, halo, C₁-C₆-haloalkyl, cyano, hydroxy, nitro, amino, —C(O)NR^{A}R^{B}, —ONR^{A}R^{B}, —O—C(O)NR^{A}R^{B}, —SO₂NR^{A}R^{B};
R^{A} and R^{B} are each independently selected from among hydrogen, C₁-C₆ alkyl, and C₃-C₆ cycloalkyl;

m and n are each independently 0-3; and

pharmaceutically acceptable salts, pharmaceutically acceptable N-oxides, pharmaceutically active metabolites, pharmaceutically acceptable prodrugs, or pharmaceutically acceptable solvates thereof.

In another embodiment is a method for determining an effective dose of a composition for increasing endogenous levels of anandamide in a subject wherein the alkylcarbamic acid aryl ester of Formula (I) has the structure according to compound KDS-4103:

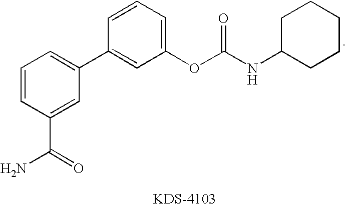

In a further embodiment is a method for determining an effective dose of a composition for increasing endogenous levels of anandamide in a subject, wherein the fatty acid amide hydrolase inhibitor is an alkylcarbamic acid aryl ester of Formula (IIa): 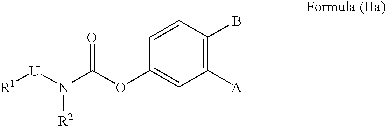
wherein:
R¹ is substituted or unsubstituted C₃-C₉ alkyl (including linear, branched, cyclic alkyl groups and combinations thereof);
R² is H or an optionally substituted alkyl;
U is a bond or CH₂;
one of A or B is (CH₂)_{q}C(O)-alkyl, (CH₂)_{q}C(O)—N(R²)₂ and the other is H, alkyl, or heteroalkyl, q is 0, 1, 2, 3, or 4;
or A and B together form an optionally substituted C(O)—(CH₂)_{q}— moiety, wherein q is 1, 2, 3 or 4;
or A and B together form an optionally substituted heteroaromatic group comprising at least one N, NR², S, or O group;
or A and B together form an optionally substituted non-aromatic or aromatic carbocycle group;
or A and B together form an optionally substituted oxo-substituted heterocycle;
or A and B are each independently selected from among H, an optionally substituted alkyl, an optionally substituted heteroalkyl, an optionally substituted heterocyclic group, an optionally substituted aryl group, an optionally substituted heteroaryl group, an optionally substituted ketoalkyl, an optionally substituted amide, and an optionally substituted ketoheteroalkyl;
one of A or B is -L-G and the other is selected from among H and an optionally substituted C₁-C₆ alkyl; or
L is a bond, or an optionally substituted group selected from among C₁-C₆ alkylene, C₁-C₆ heteroalkylene, C₁-C₆ ketoalkylene, a monosaccharide, a disaccharide, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)—, and —NR⁹C(═NR¹⁰)N(R⁹)—(CH₂)ₙ—;
G is H, tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O—(CH₂—CH₂—O)_{q}H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H₃, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —(OP(═O)OH)ₙOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)YOH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁹, —CN, and —NO₂;
n is 1, 2, 3, or 4; x is 1, 2, or 3; y is 0, 1, or 2; and

pharmaceutically acceptable salts, pharmaceutically acceptable N-oxides, pharmaceutically active metabolites, pharmaceutically acceptable prodrugs, or pharmaceutically acceptable solvates thereof.

In one aspect is a method for determining an effective dose of a composition for inhibiting fatty acid amide hydrolase activity in vivo, the method comprising:

determining a level of at least one fatty acid amide in a biological sample obtained from the subject, wherein prior to determining the level, the subject has been administered a dose of a fatty acid amide hydrolase inhibitor;

comparing the level of the at least one fatty acid amide to a pre-determined value; and

indicating that the test dose is effective when the level of the at least one fatty acid amide in the first subject is higher than the pre-determined value.

In one embodiment, is a method for determining an effective dose of a composition for inhibiting fatty acid amide hydrolase activity in vivo, wherein the biological sample is plasma, whole blood, saliva, serum, or cerebrospinal fluid. In another embodiment, is a method for determining an effective dose of a composition for inhibiting fatty acid amide hydrolase activity in vivo, wherein the biological sample is plasma. In a further embodiment, is a method for determining an effective dose of a composition for inhibiting fatty acid amide hydrolase activity in vivo, wherein the biological sample is whole blood. In yet a further embodiment, is a method for determining an effective dose of a composition for inhibiting fatty acid amide hydrolase activity in vivo, wherein the biological sample is saliva.

In one embodiment, is a method for determining an effective dose of a composition for inhibiting fatty acid amide hydrolase activity in vivo, wherein the fatty acid amide is oleoylethanolamide.

In another embodiment, is a method for determining an effective dose of a composition for inhibiting fatty acid amide hydrolase activity in vivo, wherein the fatty acid amide is palmitoylethanolamide. In a further embodiment, is a method for determining an effective dose of a composition for inhibiting fatty acid amide hydrolase activity in vivo, wherein the fatty acid amide is stearoylethanolamide. In one embodiment, is a method for determining an effective dose of a composition for inhibiting fatty acid amide hydrolase activity in vivo, wherein the fatty acid hydrolase inhibitor is administered orally.

In one embodiment, is a method for determining an effective dose of a composition for inhibiting fatty acid amide hydrolase activity in vivo, wherein the test dose is concluded to be effective when the level of the at least one fatty acid amide at the second time point is at least about 50% greater than the pre-determined value. In one embodiment, is a method for determining an effective dose of a composition for inhibiting fatty acid amide hydrolase activity in vivo, wherein the dose is concluded to be effective when the level of the at least one fatty acid amide at the second time point is at least about 90% of its saturation value. In one embodiment, is a method for determining an effective dose of a composition for inhibiting fatty acid amide hydrolase activity in vivo, wherein the dose is concluded to be effective when the level of the at least one fatty acid amide at the second time point is saturated. In one embodiment, is a method for determining an effective dose of a composition for inhibiting fatty acid amide hydrolase activity in vivo, wherein the first subject is a human. In one embodiment, is a method for determining an effective dose of a composition for inhibiting fatty acid amide hydrolase activity in vivo, wherein the first subject is a non-human primate. In one embodiment, is a method for determining an effective dose of a composition for inhibiting fatty acid amide hydrolase activity in vivo, wherein the first subject suffers from a psychiatric, neurological, neurodegenerative, or metabolic disorder. In one embodiment, is a method for determining an effective dose of a composition for inhibiting fatty acid amide hydrolase activity in vivo, wherein the subject is suffering from a metabolic disorder. In one embodiment, is a method for determining an effective dose of a composition for inhibiting fatty acid amide hydrolase activity in vivo, further comprising performing a diagnostic evaluation of the subject before and after administering the test composition.

In one embodiment, is a method for determining an effective dose of a composition for inhibiting fatty acid amide hydrolase activity in vivo, wherein the fatty acid amide hydrolase inhibitor is an alkylcarbamic acid aryl ester of Formula (I): 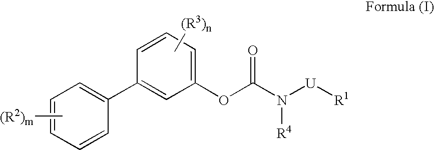
wherein:
R¹ is selected from among C₁-C₈ alkyl, C₁-C₄alkyl-(C₃-C₈cycloalkyl), and C₃-C₈ cycloalkyl (e.g., cyclohexyl); R⁴ is H or alkyl;
R² and R³ are each independently selected from among H, C₁-C₄ alkyl, C₂-C₄ alkenyl, C₂-C₄ alkynyl, C₃-C₆ cycloalkyl, C₁-C₄alkyl-(C₃-C₆cycloalkyl), aryl, substituted aryl, arylalkyl, —C(O)R^{A}, hydroxy-(C₁-C₆ alkyl), amino-(C₁-C₆ alkyl), —CH₂—NR^{A}R^{B}, —O—(C₁-C₄), aryloxy, halo, C₁-C₆-haloalkyl, cyano, hydroxy, nitro, amino, —C(O)NR^{A}R^{B}, —ONR^{A}R^{B}, —O—C(O)NR^{A}R^{B}, —SO₂NR^{A}R^{B};
R^{A} and R^{B} are each independently selected from among hydrogen, C₁-C₆ alkyl, and C₃-C₆ cycloalkyl;

m and n are each independently 0-3; and

pharmaceutically acceptable salts, pharmaceutically acceptable N-oxides, pharmaceutically active metabolites, pharmaceutically acceptable prodrugs, or pharmaceutically acceptable solvates thereof.

In another embodiment is a method for determining an effective dose of a composition for inhibiting fatty acid amide hydrolase activity in vivo, wherein the alkylcarbamic acid aryl ester of Formula (I) has the structure according to compound KDS-4103:

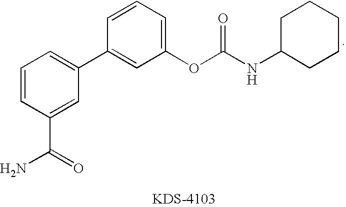

In a further embodiment is a method for determining an effective dose of a composition for inhibiting fatty acid amide hydrolase activity in vivo, wherein the fatty acid amide hydrolase inhibitor is an alkylcarbamic acid aryl ester of Formula (IIa): 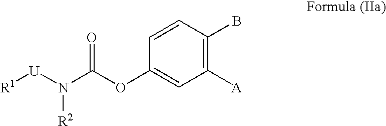
wherein:
R¹ is substituted or unsubstituted C₃-C₉ alkyl (including linear, branched, cyclic alkyl groups and combinations thereof);
R² is H or an optionally substituted alkyl;
U is a bond or CH₂;
one of A or B is (CH₂)_{q}C(O)-alkyl, (CH₂)_{q}C(O)—N(R²)₂ and the other is H, alkyl, or heteroalkyl, q is 0, 1, 2, 3, or 4;
or A and B together form an optionally substituted C(O)—(CH₂)_{q}— moiety, wherein q is 1, 2, 3 or 4;
or A and B together form an optionally substituted heteroaromatic group comprising at least one N, NR², S, or O group;
or A and B together form an optionally substituted non-aromatic or aromatic carbocycle group;
or A and B together form an optionally substituted oxo-substituted heterocycle;
or A and B are each independently selected from among H, an optionally substituted alkyl, an optionally substituted heteroalkyl, an optionally substituted heterocyclic group, an optionally substituted aryl group, an optionally substituted heteroaryl group, an optionally substituted ketoalkyl, an optionally substituted amide, and an optionally substituted ketoheteroalkyl;
one of A or B is -L-G and the other is selected from among H and an optionally substituted C₁-C₆ alkyl; or
L is a bond, or an optionally substituted group selected from among C₁-C₆ alkylene, C₁-C₆ heteroalkylene, C₁-C₆ ketoalkylene, a monosaccharide, a disaccharide, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O—(CH₂—CH₂—O)_{q}H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H₃, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —(OP(═O)OH)ₙOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁹, —CN, and —NO₂;
n is 1, 2, 3, or 4; x is 1, 2, or 3; y is 0, 1, or 2; and

pharmaceutically acceptable salts, pharmaceutically acceptable N-oxides, pharmaceutically active metabolites, pharmaceutically acceptable prodrugs, or pharmaceutically acceptable solvates thereof.

In one aspect is a method for optimizing therapeutic efficacy for treatment of anxiety, depression, pain, or a metabolic disorder, the method comprising determining a level of at least one fatty acid amide in a biological sample obtained from a subject who has been administered a dose of a drug providing an alkylcarbamic acid aryl ester of Formula (I): 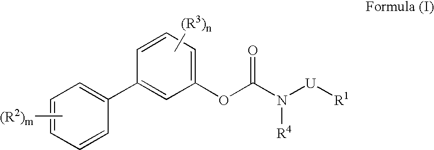
wherein:
R¹ is selected from among C₁-C₈ alkyl, C₁-C₄alkyl-(C₃-C₈cycloalkyl), and C₃-C₈ cycloalkyl (e.g., cyclohexyl); R⁴ is H or alkyl;
R² and R³ are each independently selected from among H, C₁-C₄ alkyl, C₂-C₄ alkenyl, C₂-C₄ alkynyl, C₃-C₆ cycloalkyl, C₁-C₄alkyl-(C₃-C₆cycloalkyl), aryl, substituted aryl, arylalkyl, —C(O)R^{A}, hydroxy-(C₁-C₆ alkyl), amino-(C₁-C₆ alkyl), —CH₂—NR^{A}R^{B}, —O—(C₁-C₄), aryloxy, halo, C₁-C₆-haloalkyl, cyano, hydroxy, nitro, amino, —C(O)NR^{A}R^{B}, —ONR^{A}R^{B}, —O—C(O)NR^{A}R^{B}, —SO₂NR^{A}R^{B};
R^{A} and R^{B} are each independently selected from among hydrogen, C₁-C₆ alkyl, and C₃-C₆ cycloalkyl;

m and n are each independently 0-3; and

indicating a need to increase the amount of the drug subsequently administered to the subject for a level of the at least one fatty acid amide less than 50% of a pre-determined value.

In one embodiment is a method for optimizing therapeutic efficacy for treatment of anxiety, depression, pain, or a metabolic disorder, wherein the at least one fatty acid amide is oleoylethanolamide. In another embodiment is a method for optimizing therapeutic efficacy for treatment of anxiety, depression, pain, or a metabolic disorder, wherein the at least one fatty acid amide is palmitoylethanolamide. In yet another embodiment is a method for optimizing therapeutic efficacy for treatment of anxiety, depression, pain, or a metabolic disorder, wherein the at least one fatty acid amide is stearoylethanolamide. In one embodiment is a method for optimizing therapeutic efficacy for treatment of anxiety, depression, pain, or a metabolic disorder, wherein the subject is a human. In another embodiment is a method for optimizing therapeutic efficacy for treatment of anxiety, depression, pain, or a metabolic disorder, wherein the subject is a non-human primate. In yet another embodiment is a method for optimizing therapeutic efficacy for treatment of anxiety, depression, pain, or a metabolic disorder, wherein the biological sample is plasma, saliva, whole blood, serum, or cerebrospinal fluid. In a further embodiment is a method for optimizing therapeutic efficacy for treatment of anxiety, depression, pain, or a metabolic disorder, wherein the biological sample is plasma. In yet a further embodiment is a method for optimizing therapeutic efficacy for treatment of anxiety, depression, pain, or a metabolic disorder, wherein the biological sample is whole blood. In yet a further embodiment is a method for optimizing therapeutic efficacy for treatment of anxiety, depression, pain, or a metabolic disorder, wherein the biological sample is saliva. In one embodiment is a method for optimizing therapeutic efficacy for treatment of anxiety, depression, pain, or a metabolic disorder, wherein the drug is administered orally. In one embodiment is a method for optimizing therapeutic efficacy for treatment of pain wherein the pain is selected from the group consisting of nociceptive pain, neuropathic pain, inflammatory pain, non-inflammatory pain, painful hemorrhagic cystitis, pain associated with the herpes virus, pain associated with diabetes, peripheral neuropathic pain, peri-operative pain, cancer pain, pain and spasticity associated with multiple sclerosis, central pain, deafferentiation pain, chronic nociceptive pain, stimulus of nociceptive receptors, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

In one aspect is a method for optimizing therapeutic efficacy for treatment of anxiety, depression, pain, or a metabolic disorder, the method comprising determining a level of at least one fatty acid amide in a biological sample obtained from a subject who has been administered a dose of a drug providing an alkylcarbamic acid aryl ester of Formula (IIa): 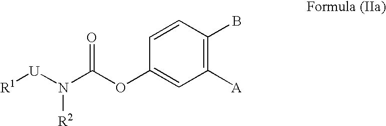
wherein:
R¹ is substituted or unsubstituted C₃-C₉ alkyl (including linear, branched, cyclic alkyl groups and combinations thereof);
R² is H or an optionally substituted alkyl;
U is a bond or CH₂;
one of A or B is (CH₂)_{q}C(O)-alkyl, (CH₂)_{q}C(O)—N(R²)₂ and the other is H, alkyl, or heteroalkyl, q is 0, 1, 2, 3, or 4;
or A and B together form an optionally substituted C(O)—(CH₂)_{q}— moiety, wherein q is 1, 2, 3 or 4;
or A and B together form an optionally substituted heteroaromatic group comprising at least one N, NR², S, or O group;
or A and B together form an optionally substituted non-aromatic or aromatic carbocycle group;
or A and B together form an optionally substituted oxo-substituted heterocycle;
or A and B are each independently selected from among H, an optionally substituted alkyl, an optionally substituted heteroalkyl, an optionally substituted heterocyclic group, an optionally substituted aryl group, an optionally substituted heteroaryl group, an optionally substituted ketoalkyl, an optionally substituted amide, and an optionally substituted ketoheteroalkyl;
one of A or B is -L-G and the other is selected from among H and an optionally substituted C₁-C₆ alkyl; or
L is a bond, or an optionally substituted group selected from among C₁-C₆ alkylene, C₁-C₆ heteroalkylene, C₁-C₆ ketoalkylene, a monosaccharide, a disaccharide, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O—(CH₂—CH₂—O)_{q}—H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H₃, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —(OP(═O)OH)ₙOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁹, —CN, and —NO₂;
n is 1, 2, 3, or 4; x is 1, 2, or 3; y is 0, 1, or 2;
   pharmaceutically acceptable salts, pharmaceutically acceptable N-oxides, pharmaceutically active metabolites, pharmaceutically acceptable pro drugs, or pharmaceutically acceptable solvates thereof; and

indicating a need to increase the amount of the drug subsequently administered to the subject for a level of the at least one fatty acid amide less than 50% of a pre-determined value.

In some embodiments, FAAH inhibitors, e.g., those of Formula (II) above, are ionizable at physiological pH, and are therefore less likely to cross the blood brain barrier into the central nervous system compared to FAAH inhibitors that lack groups that are ionizable at physiological pH. Such FAAH inhibitors are particularly useful when it is desirable to avoid psychotropic effects caused by FAAH inhibition in the central nervous system.

FAAH inhibitor compositions that can be used with the methods described herein are described in U.S. patent application Ser. Nos. 10/681,858, 60/755,035; U.S. Pat. Nos. 6,462,054 and 6,891,043; and PCT Nos. WO04020430, WO04067498, WO04099176, WO05033066, WO02087569, WO03065989, WO9749667, WO9926584, WO04033652, WO06044617; which are incorporated by reference for this purpose.

Examples of Pharmaceutical Compositions and Methods of Administration

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincoff Williams & Wilkins 1999), herein incorporated by reference in their entirety.

Provided herein are pharmaceutical compositions that include a compound described herein, such as, compounds of Formula (I) compounds of Formula (II), or Formula (IIa) and a pharmaceutically acceptable diluent(s), excipient(s), or carrier(s). In addition, the compounds described herein can be administered as pharmaceutical compositions in which compounds described herein are mixed with other active ingredients, as in combination therapy. In some embodiments, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

In certain embodiments, compositions may also include one or more pH adjusting agents or buffering agents, including organic acids such as acetic, citric, lactic, ascorbic, tartaric, maleic, malonic, fumaric, glycolic, succinic, propionic, and methane sulfonic acid; and mineral acids such as phosphoric, hydrobromic, sulfuric, boric, and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

A pharmaceutical composition, as used herein, refers to a mixture of a compound described herein, such as, for example, compounds of Formula (I), compounds of Formula (II), or Formula (IIa) with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound described herein, such as, for example, a compound of Formula (I), a compound of Formula (II), or a compound of Formula (IIa) as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein.

“Bioavailability” refers to the percentage of the weight of compounds disclosed herein, such as, compounds of Formula (I), a compound of Formula (II), or a compound of Formula (IIa), dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which compounds disclosed herein, such as, compounds of Formula (I), a compound of Formula (II), or a compound of Formula (IIa), are absorbed into the general circulation when the pharmaceutical composition is taken orally as compared to intravenous injection.

“Blood plasma concentration” refers to the concentration of compounds disclosed herein, such as, compounds of Formula (I), a compound of Formula (II), or a compound of Formula (IIa), in the plasma component of blood of a subject. It is understood that the plasma concentration of compounds of Formula (I), a compound of Formula (II), or a compound of Formula (IIa) may vary significantly between subjects, due to variability with respect to metabolism and/or possible interactions with other therapeutic agents. In accordance with one embodiment disclosed herein, the blood plasma concentration of the compounds of Formula (I), a compound of Formula (II), or a compound of Formula (IIa) may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of a compound of Formula (I), a compound of Formula (II), or a compound of Formula (IIa) may vary from subject to subject.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Steady state,” as used herein, is when the amount of drug administered is equal to the amount of drug eliminated within one dosing interval resulting in a plateau or constant plasma drug exposure.

Dosage Forms

The compositions described herein can be formulated for administration to a subject via any conventional means including, but not limited to, oral, parenteral (e.g., intravenous, subcutaneous, intrathecal, or intramuscular), buccal, intranasal, epidural, pulmonary, local, rectal or transdermal administration routes. As used herein, the term “subject” is used to mean an animal, preferably a mammal, including a human or non-human. The terms patient and subject may be used interchangeably.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., “The Theory and Practice of Industrial Pharmacy” (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound of Formula (I), a compound of Formula (II), or a compound of Formula (IIa), and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof, as described in the standard reference Gennaro, A. R. et al., Remington: The Science and Practice of Pharmacy (20th Edition, Lippincott Williams & Wilkins, 2000, see especially Part 5: Pharmaceutical Manufacturing).

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition to the particles of compound of Formula (I), a compound of Formula (II), or a compound of Formula (IIa), the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

Examples of Methods of Dosing and Treatment Regimens

The compounds described herein can be used in the preparation of medicaments for the inhibition of fatty acid amide hydrolase, or for the treatment of diseases or conditions that would benefit, at least in part, from inhibition of fatty acid amide hydrolase. In addition, a method for treating any of the diseases or conditions described herein in a subject in need of such treatment, involves administration of pharmaceutical compositions containing at least one compound of Formula (I), a compound of Formula (II), or a compound of Formula (IIa) described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In one embodiment, a compound of Formula (I), a compound of Formula (II), or a compound of Formula (IIa) described herein, are useful in the treatment of a variety of painful syndromes, diseases, disorders, and/or conditions, including but not limited to those characterized by non-inflammatory pain, inflammatory pain, peripheral neuropathic pain, peri-operative pain, cancer pain, pain and spasticity associated with multiple sclerosis, central pain, deafferentiation pain, chronic nociceptive pain, stimulus of nociceptive receptors, phantom and transient acute pain. In one embodiment, is a method for treating pain comprising administering the alkylcarbamic acid aryl esters of Formula (I) prepared by the process described herein.

The compounds described herein may also be useful in the treatment of other disorders such as spasticity, glaucoma, nausea, emesis, loss of appetite, sleep disturbances, respiratory disorders, allergies, epilepsy, migraine, cardiovascular disorders, anxiety, traumatic brain injury and stroke.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, preferably 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

In some embodiments, the daily dosages appropriate for the compounds described herein to alleviate the symptoms described herein are from about 0.001 to about 50 mg/kg per body weight. In other embodiments, the daily dosages appropriate for the compounds described herein are from about 0.01 to about 20 mg/kg per body weight. In further embodiments, the daily dosages appropriate for the compounds described herein described herein are from about 0.01 to about 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to about 50 mg active ingredient. The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Examples of Combination Treatments

The compositions and methods described herein may also be used in conjunction with other well known therapeutic reagents that are selected for their particular usefulness against the condition that is being treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In addition, the compounds described herein also may be used in combination with procedures that may provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a mutant gene that is known to be correlated with certain diseases or conditions.

### EXAMPLES

The following specific examples are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

### Example 1

### Plasma Levels of FAAs are Highly Correlated with FAAH Inhibition in the Brain

Due to a poor correlation between plasma exposure of KDS-4103 and in vivo activity, we sought a peripheral bio-marker that correlated with FAAH inhibition in the brain. Anandamide was considered as a possible bio-marker in plasma since changes in anandamide levels appear to mediate the pharmacological effects of FAAH inhibitors. However, circulating levels of anandamide in rodents and humans have typically been found at extremely low levels in vivo and, in most cases, were below the limit of quantitation of our LC-MS/MS methods (0.1 ng/ml).

We hypothesized that plasma levels of more abundant FAEs, such as OEA and PEA, would correlate with FAAH inhibition in the brain such that escalating doses of a FAAH inhibitor would be paralleled by increased levels of these FAEs in plasma. Moreover, complete FAAH inhibition was expected to cause a maximal increase in FAE levels with no further increases observed even at greater doses of FAAH inhibitor. Thus, we examined (in rats) the relationship between FAAH inhibition in the brain and levels of OEA and PEA in plasma.

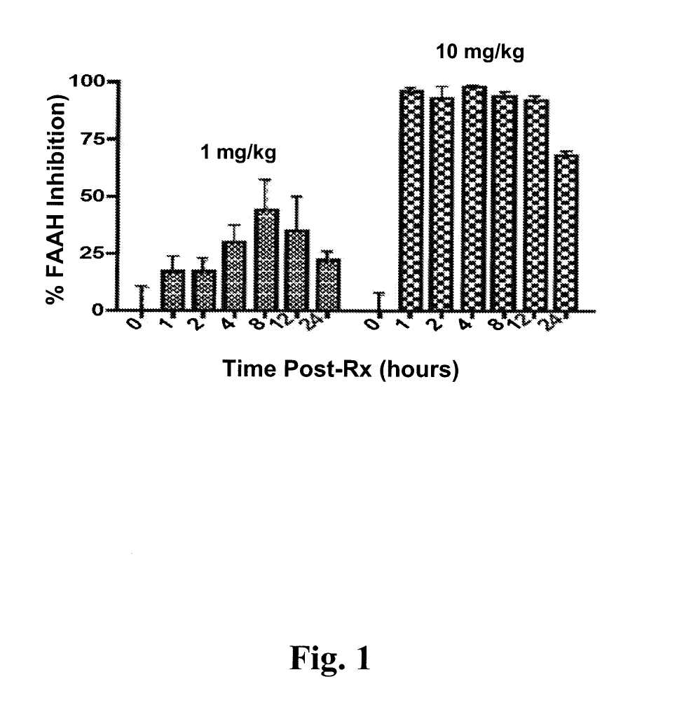

Groups of animals were orally administered KDS-4103, an irreversible inhibitor of FAAH, at low (1 mg/kg) and high 10 (mg/kg) doses. At 0, 1, 2, 4, 8, 12, and 24 hours post-administration, animals were sacrificed to collect serum and brain tissue samples. For each time point, FAAH activity was determined in brain tissue homogenates by essentially the same method as that described in Fegley et al. (2005), J. Pharm. and Exp. Ther., 313:352-358. We observed complete FAAH inhibition at 10 mg/kg and partial FAAH inhibition at 1 mg/kg (FIG. 1).

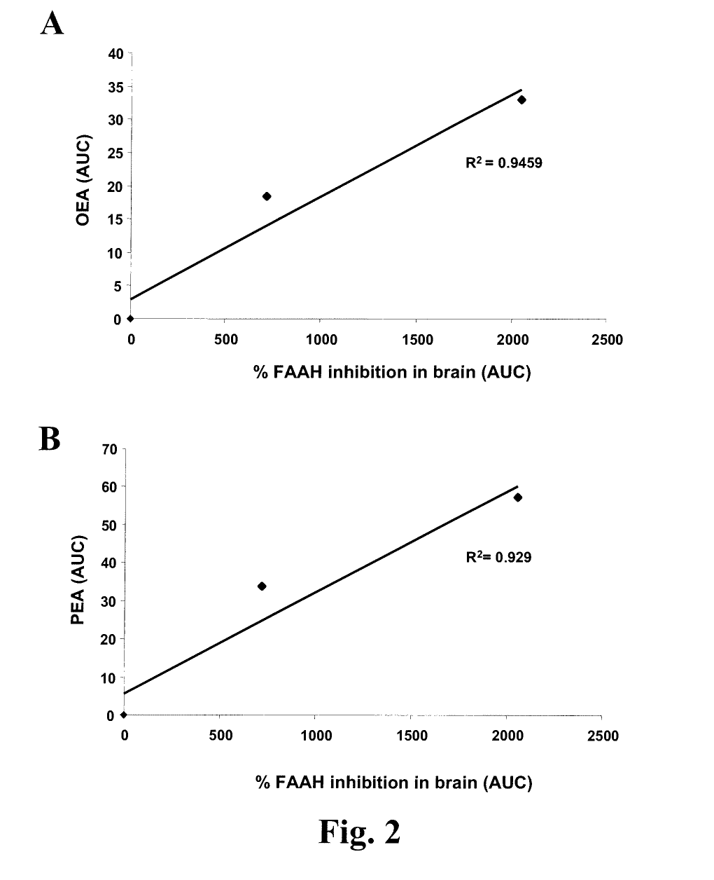

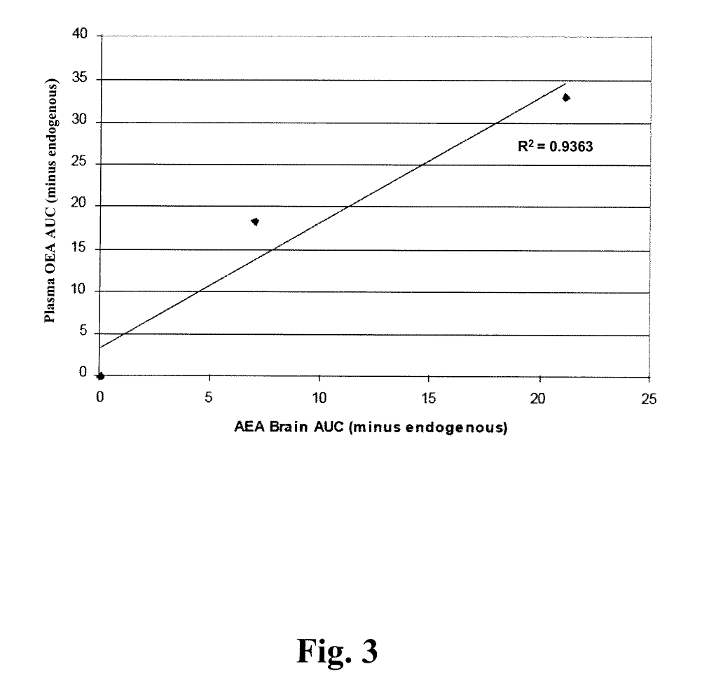

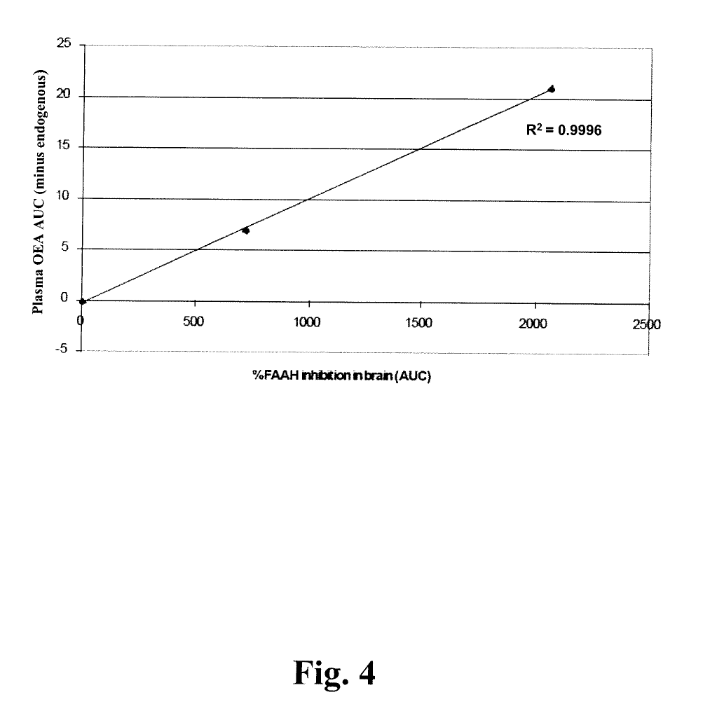

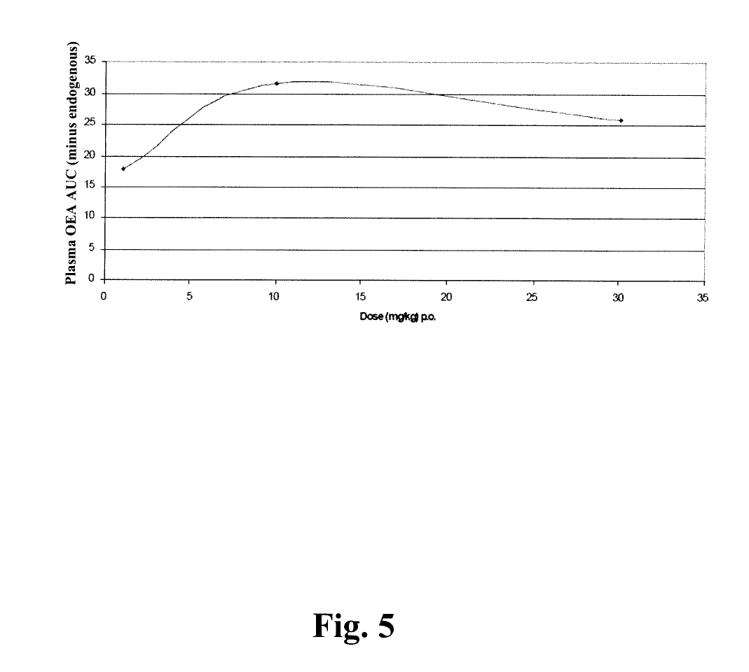

Serum OEA and PEA levels were determined by LC-MS/MS. As shown in FIGS. 2 (A-B), plasma levels of both OEA and PEA were highly correlated with FAAH inhibition in the brain (R²=0.9459 and 0.929, respectively). In addition, changes in OEA plasma levels correlated with changes in anandamide (AEA) levels in the brain (FIG. 3; R²=0.9363), and changes in AEA brain levels correlated with brain FAAH inhibition (FIG. 4; R²=0.9996). Furthermore, increasing the dose of KDS-4103 to 30 mg/kg, p.o. (i.e., at least three-fold the dose required to maximally inhibit brain FAAH activity) did not increase plasma levels of OEA above those observed at 10 mg/kg, p.o. (FIG. 5). For these and other figures, AUC values are presented as area under the curve calculations for 1) levels of OEA or PEA minus endogenous starting levels and 2) percent inhibition for FAAH activity (AUCs are 0-24 hrs). AUCs were determined as the sum of the area under the plasma concentration curve of the analyte measured (minus the endogenous level), or as a % FAAH inhibition versus time curve from time zero to the last time point collected by the trapezoidal rule.

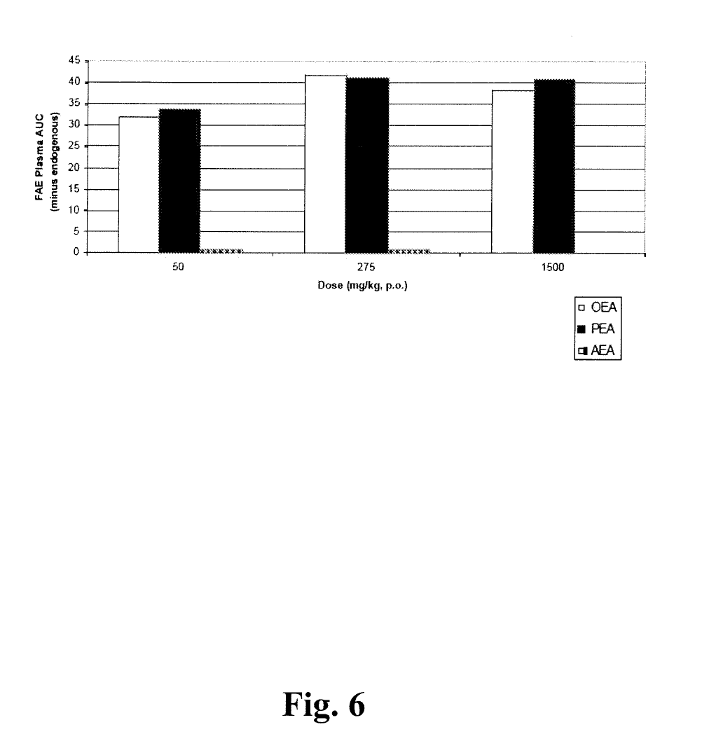

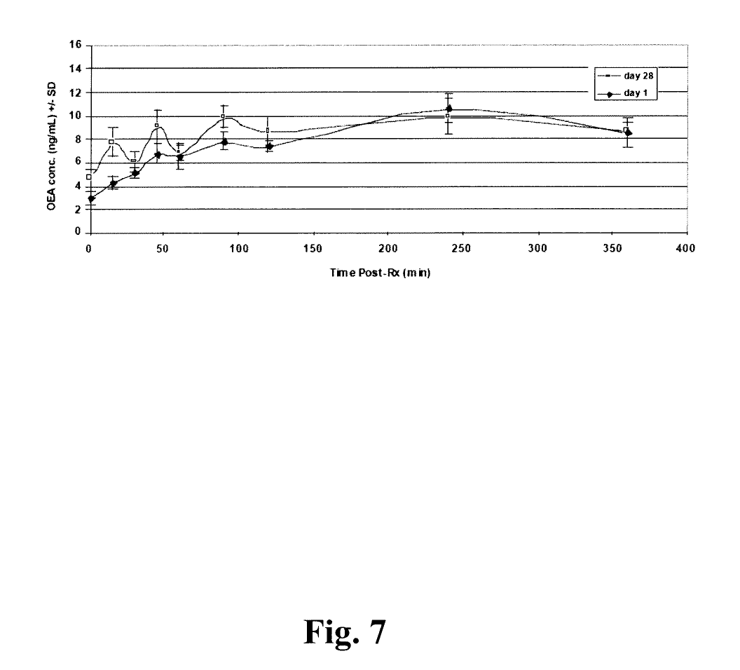

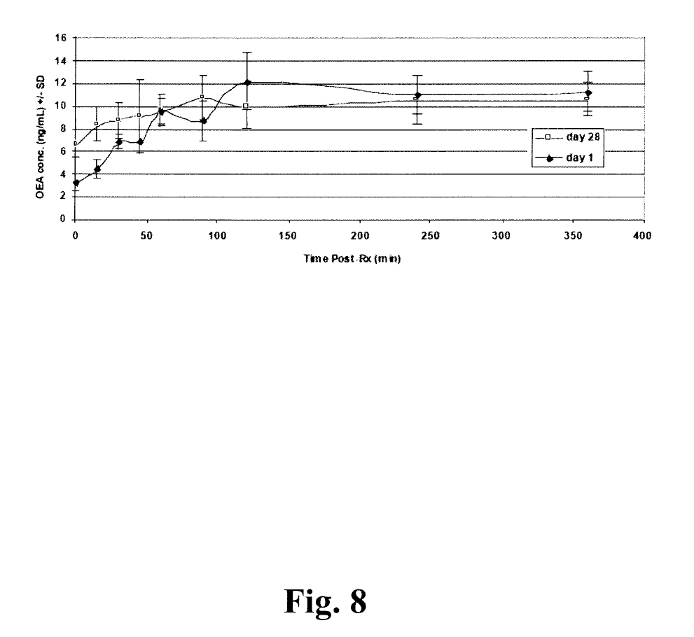

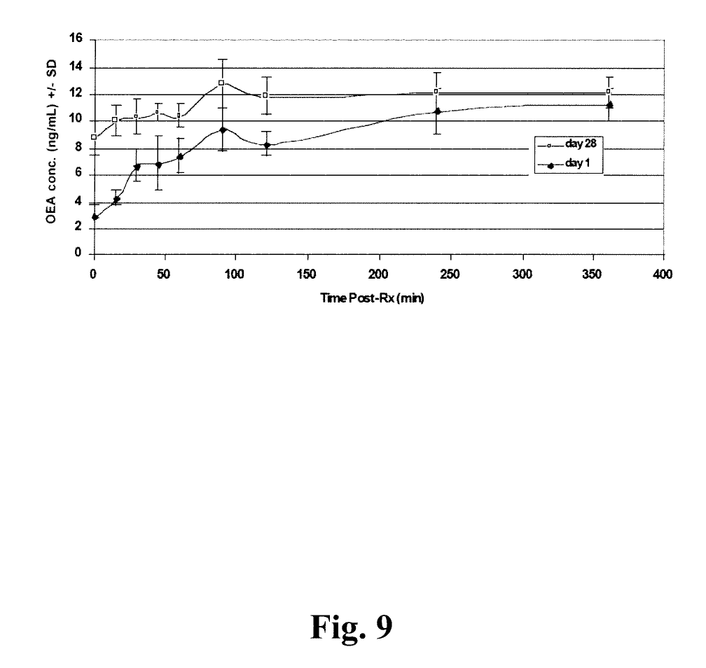

We carried out toxicokinetic (TK) studies in rodents. OEA, PEA, and AEA were quantitated simultaneously with KDS-4103 over a period of 28 days. AEA was included in this assay to examine whether basal or elevated levels of AEA could be detected. The results of the TK studies demonstrated that OEA and PEA levels were consistently detectable in plasma, while AEA levels were generally below the limit of quantitation. Increases in OEA and PEA levels were similar across the evaluated dose range of 50, 275, and 1500 mg/kg (FIG. 6), indicating that FAAH inhibition was complete across all doses. Following 28-days of treatment, maximum levels of OEA and PEA in plasma following KDS-4103 dosing were similar to maximum levels after Day 1 treatments (FIGS. 7-9).

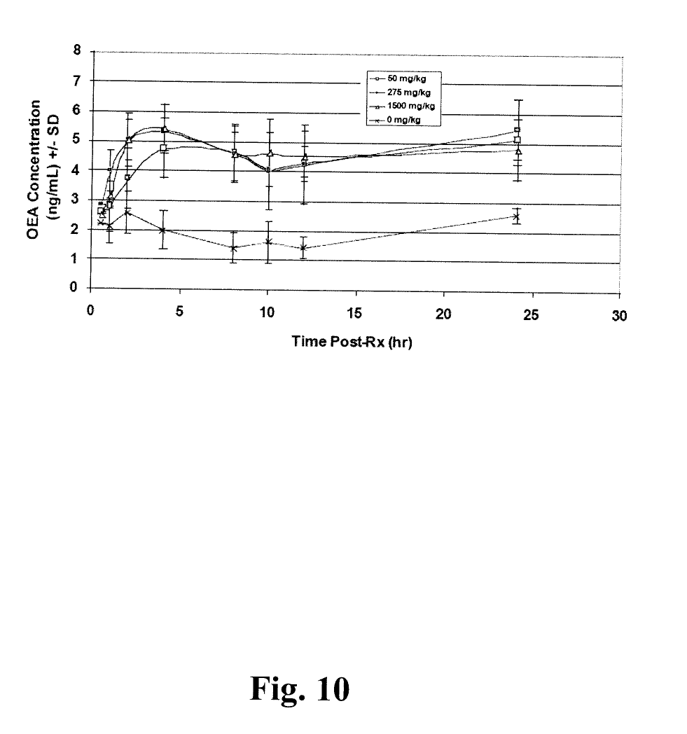

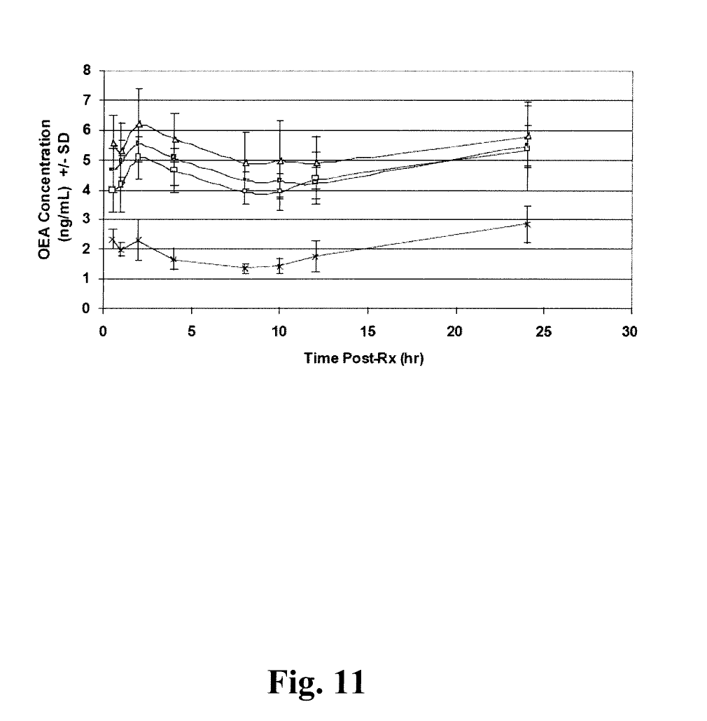

Following the rodent TK studies, we carried out 28-day monkey TK studies in which OEA levels were quantitated in parallel with KDS-4103 in. As observed in the rat, changes in OEA plasma levels in the monkey were similar among doses of KDS-4103 (50, 275, and 1500 mg/kg, p.o.) on Day 1 and Day 28 of treatments (FIGS. 10 and 11). These data were especially compelling since plasma from each animal, including vehicle controls, was sampled at each time point. Despite variations in OEA levels over time, all doses of KDS-4103 consistently resulted in 2- to 3-fold increases in plasma OEA levels compared to vehicle. In KDS-4103-treated animals OEA levels were elevated from 1 hour through 24 hours post-dose on Day 1 and from 0.25 hours (first time point) through 24 hours post dose on Day 28 compared to vehicle-treated controls.

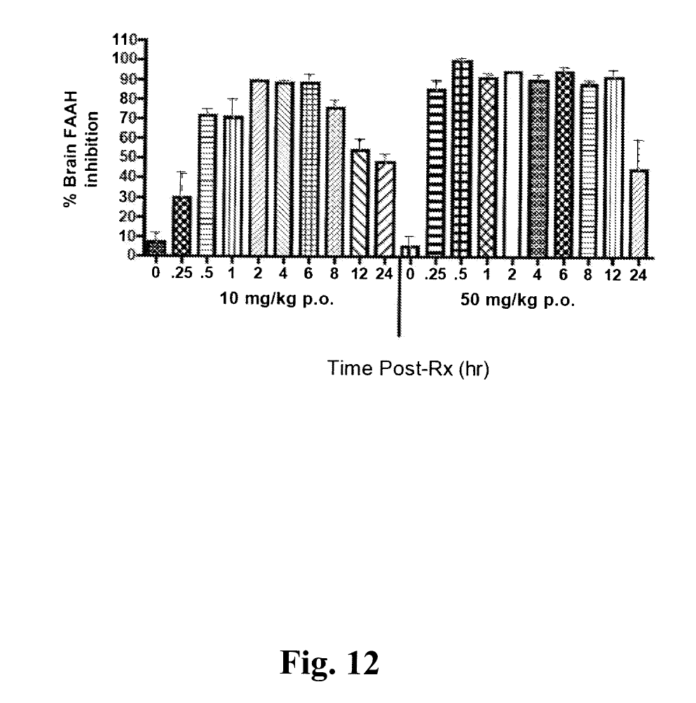

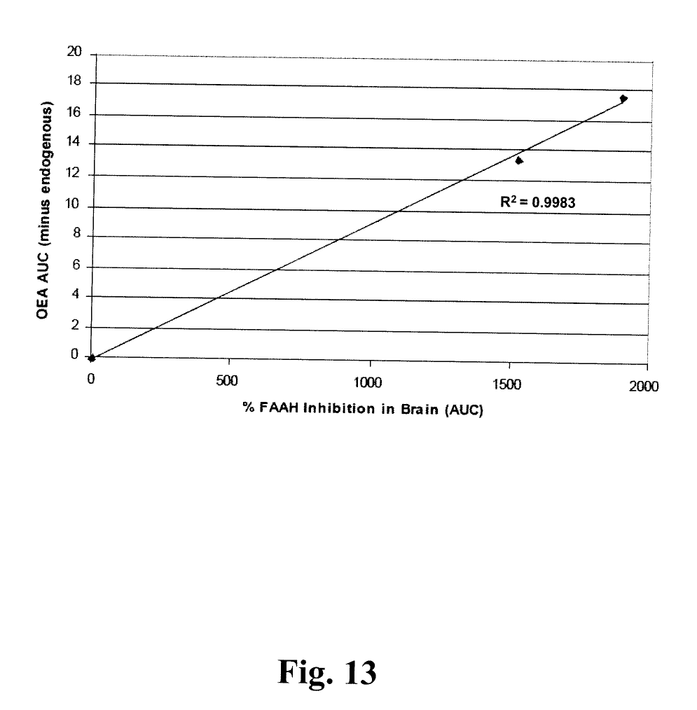

Finally, the correlation between increases in plasma OEA levels and brain FAAH Activity was also evaluated in an escalating oral dose study in mice. Inhibition of FAAH activity was maximal at 50 mg/kg p.o. and partial 10 mg/kg p.o. (FIG. 12). Increases in plasma OEA levels directly correlated with brain FAAH inhibition (FIG. 13).

Based on these data it was predicted that maximal brain FAAH inhibition would be achieved at a KDS-4103 dose that results in maximal increases in plasma OEA levels.

### Example 2

### KDS-4103 Causes Prolonged Elevation of Plasma OEA Levels in Primates

We established a clear relationship between inhibition of FAAH activity in brain and elevation of plasma FAA levels by administering KDS-4103 in rats. Thus, we wished to examine the ability of KDS-4103 to inhibit FAAH in primates vis-a-vis elevation of OEA levels in plasma.

KDS-4103 at doses of 50-1500 mg/kg, or vehicle, was administered orally to cynomolgus monkeys. Afterwards, a time course of plasma OEA levels was determined for each subject by obtaining a plasma sample at 0.5, 1, 2, 4, 8, 12, and 24 hours post-administration. As shown in FIG. 2, in subjects administered each dose of KDS-4103, plasma OEA levels were clearly elevated two hours after administration, peaked at four hours, and remained elevated at all subsequent time points examined. On the basis of these data, we concluded that KDS-4103 is highly effective for inhibiting FAAH activity in primates.

Throughout the specification, claims and accompanying figures, a number of embodiments of the invention have been described. Nevertheless, it will be understood that various modifications may be made without departing from the spirit and scope of the invention. Accordingly, other embodiments are within the scope of the following claims.

What is claimed is:

1. A method for determining an effective dose of a composition for increasing endogenous levels of anandamide in a subject, the method comprising:
determining a level of at least one fatty acid amide other than anandamide in a biological sample obtained from the subject at a first time point;
determining a level of the at least one fatty acid amide in a biological sample obtained from the subject at a second time point, wherein prior to the second time point the subject has been administered a dose of a fatty acid amide hydrolase inhibitor, and
indicating that the dose is effective when the level of the at least one fatty acid amide is greater in the biological sample obtained at the second time point than at the first time point.

2. The method of claim 1, wherein the biological sample obtained at the first and second time points is plasma, whole blood, serum, saliva, or cerebrospinal fluid.

3. The method of claim 2, wherein the biological sample obtained at the first and second time points is plasma.

4. The method of claim 2, wherein the biological sample obtained at the first and second time points is whole blood.

5. The method of claim 2, wherein the biological sample obtained at the first and second time points is saliva.

6. The method of claim 1, wherein the fatty acid amide hydrolase inhibitor is administered orally.

7. The method of claim 1, further indicating that the dose of fatty acid amide hydrolase inhibitor is effective when the level of the at least one fatty acid amide at the second time point is at least about 50% greater than at the first time point.

8. The method of claim 1, further indicating that the dose of fatty acid amide hydrolase inhibitor is effective when the level of the at least one fatty acid amide at the second time point is at least about 90% of its saturation value.

9. The method of claim 1, further indicating that the dose of fatty acid amide hydrolase inhibitor is effective when the level of the at least one fatty acid amide at the second time point is saturated.

10. The method of claim 1, wherein the subject is suffering from a psychiatric, neurological, neurodegenerative, painful, or metabolic disorder.

11. The method of claim 10, wherein the subject is suffering from a metabolic disorder.

12. The method of claim 10, further comprising performing a diagnostic evaluation of the subject before and after administering the dose.

13. A method for determining an effective dose of a composition for inhibiting fatty acid amide hydrolase activity in vivo, the method comprising:
determining a level of at least one fatty acid amide in a biological sample obtained from a subject, wherein prior to determining the level, the subject has been administered a dose of a fatty acid amide hydrolase inhibitor; and
indicating that the dose is effective when the level of the at least one fatty acid amide in the first subject is higher than a pre-determined value.

14. The method of claim 13, wherein the dose is administered orally.

15. The method of claim 13, further indicating that the dose of fatty acid amide hydrolase inhibitor is effective when the level of the at least one fatty acid amide at the second time point is at least about 50% greater than the pre-determined value.

16. The method of claim 13, further indicating that the dose of fatty acid amide hydrolase inhibitor is effective when the level of the at least one fatty acid amide at the second time point is at least about 90% of its saturation value.

17. The method of claim 13, further indicating that the dose of fatty acid amide hydrolase inhibitor is effective when the level of the at least one fatty acid amide at the second time point is saturated.

18. The method of claim 13, wherein the first subject suffers from a psychiatric, neurological, neurodegenerative, or metabolic disorder.

19. The method of claim 18, wherein the first subject is suffering from a metabolic disorder.

20. The method of claim 18, further comprising performing a diagnostic evaluation of the subject before and after administering the test composition.

21. The method of claim 1, wherein the fatty acid amide hydrolase inhibitor is an alkylcarbamic acid aryl ester of Formula (I): 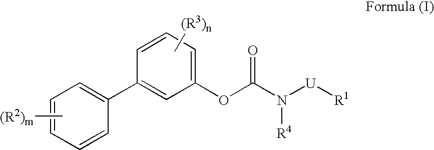
wherein:
R¹ is selected from C₁-C₈ alkyl, C₁-C₄alkyl-(C₃-C₈cycloalkyl), and C₃-C₈ cycloalkyl (e.g., cyclohexyl); R⁴ is H or alkyl;
R² and R³ are each independently selected from H, C₁-C₄ alkyl, C₂-C₄ alkenyl, C₂-C₄ alkynyl, C₃-C₆ cycloalkyl, C₁-C₄alkyl-(C₃-C₆cycloalkyl), aryl, substituted aryl, arylalkyl, —C(O)R^{A}, hydroxy-(C₁-C₆ alkyl), amino-(C₁-C₆ alkyl), —CH₂—NR^{A}R^{B}, —O—(C₁-C₄), aryloxy, halo, C₁-C₆-haloalkyl, cyano, hydroxy, nitro, amino, —C(O)NR^{A}R^{B}, —ONR^{A}R^{B}, —O—C(O)NR^{A}R^{B}, —SO₂NR^{A}R^{B};
R^{A} and R^{B} are each independently selected from hydrogen, C₁-C₆ alkyl, and C₃-C₆ cycloalkyl;
m and n are each independently 0-3; and
pharmaceutically acceptable salts, pharmaceutically acceptable N-oxides, pharmaceutically active metabolites, pharmaceutically acceptable pro drugs, or pharmaceutically acceptable solvates thereof.

22. The method of claim 21, wherein the alkylcarbamic acid aryl ester of Formula (I) has the structure according to compound KDS-4103:

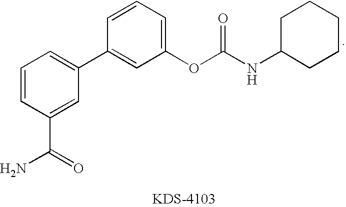

23. The method of claim 1, wherein the fatty acid amide hydrolase inhibitor is an alkylcarbamic acid aryl ester of Formula (IIa): 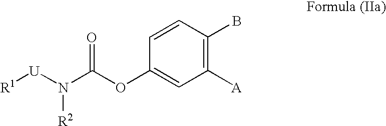
wherein:
R¹ is substituted or unsubstituted C₃-C₉ alkyl (including linear, branched, cyclic alkyl groups and combinations thereof);
R² is H or an optionally substituted alkyl;
U is a bond or CH₂;
one of A or B is (CH₂)_{q}C(O)-alkyl, (CH₂)_{q}C(O)—N(R²)₂ and the other is H, alkyl, or heteroalkyl, q is 0, 1, 2, 3, or 4;
or A and B together form an optionally substituted C(O)—(CH₂)_{q}— moiety, wherein q is 1, 2, 3 or 4;
or A and B together form an optionally substituted heteroaromatic group comprising at least one N, NR S, or O group;
or A and B together form an optionally substituted non-aromatic or aromatic carbocycle group;
or A and B together form an optionally substituted oxo-substituted heterocycle;
or A and B are each independently selected from among H, an optionally substituted alkyl, an optionally substituted heteroalkyl, an optionally substituted heterocyclic group, an optionally substituted aryl group, an optionally substituted heteroaryl group, an optionally substituted ketoalkyl, an optionally substituted amide, and an optionally substituted ketoheteroalkyl;
one of A or B is -L-G and the other is selected from among H and an optionally substituted C₁-C₆ alkyl; or
L is a bond, or an optionally substituted group selected from among C₁-C₆ alkylene, C₁-C₆ heteroalkylene, C₁-C₆ ketoalkylene, a monosaccharide, a disaccharide, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —CH₂—(O—CH₂—CH₂)_{q}—O—CH₃, —O—(CH₂—CH₂—O)_{q}—CH₃, —CH₂—(OCH₂—CH₂)_{q}—OH, —O—(CH₂—CH₂—O)_{q}—H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H₃, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —(OP(═O)OH)ₙOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NO₂;
n is 1, 2, 3, or 4; x is 1, 2, or 3; y is 0, 1, or 2; and
pharmaceutically acceptable salts, pharmaceutically acceptable N-oxides, pharmaceutically active metabolites, pharmaceutically acceptable pro drugs, or pharmaceutically acceptable solvates thereof.

24. A method for optimizing therapeutic efficacy for treatment of anxiety, depression, pain, or a metabolic disorder, the method comprising determining a level of at least one fatty acid amide in a biological sample obtained from a subject who has been administered a dose of a drug providing an alkylcarbamic acid aryl ester of Formula (I): 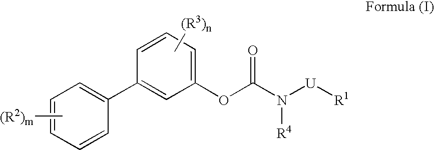
wherein:
R¹ is selected from among C₁-C₈ alkyl C₁-C₄alkyl-(C₃-C₈cycloalkyl), and C₃-C₈ cycloalkyl (e.g., cyclohexyl); R⁴ is H or alkyl;
R² and R³ are each independently selected from among H, C₁-C₄ alkyl, C₂-C₄ alkenyl, C₂-C₄ alkynyl, C₃-C₆ cycloalkyl, C₁-C₄alkyl-(C₃-C₆cycloalkyl), aryl, substituted aryl, arylalkyl, —C(O)R^{A}, hydroxy-(C₁-C₆ alkyl), amino-(C₁-C₆ alkyl), —CH₂—NR^{A}R^{B}, —O—(C₁-C₄), aryloxy, halo, C₁-C₆-haloalkyl, cyano, hydroxy, nitro, amino, —C(O)NR^{A}R^{B}, —ONR^{A}R^{B}, —O—C(O)NR^{A}R^{B}, —SO₂NR^{A}R_{B};
R^{A} and R^{B} are each independently selected from among hydrogen, C₁-C₆ alkyl, and C₃-C₆ cycloalkyl;
m and n are each independently 0-3; and
indicating a need to increase the amount of the drug subsequently administered to the subject for a level of the at least one fatty acid amide less than 50% of a pre-determined value.

25. A method for optimizing therapeutic efficacy for treatment of anxiety, depression, pain, or a metabolic disorder, the method comprising determining a level of at least one fatty acid amide in a biological sample obtained from a subject who has been administered a dose of a drug providing an alkylcarbamic acid aryl ester of Formula (IIa): 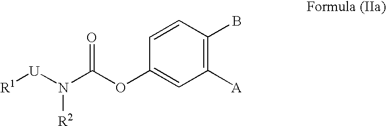
wherein:
R¹ is substituted or unsubstituted C₃-C₉ alkyl (including linear, branched, cyclic alkyl groups and combinations thereof);
R² is H or an optionally substituted alkyl;
U is a bond or CH₂;
one of A or B is (CH₂)_{q}C(O)-alkyl, (CH₂)_{q}C(O)—N(R²)₂ and the other is H, alkyl, or heteroalkyl, q is 0, 1, 2, 3, or 4;
or A and B together form an optionally substituted C(O)—(CH₂)_{q}— moiety, wherein q is 1, 2, 3 or 4;
or A and B together form an optionally substituted heteroaromatic group comprising at least one N, NR S, or O group;
or A and B together form an optionally substituted non-aromatic or aromatic carbocycle group;
or A and B together form an optionally substituted oxo-substituted heterocycle;
or A and B are each independently selected from among H, an optionally substituted alkyl, an optionally substituted heteroalkyl, an optionally substituted heterocyclic group, an optionally substituted aryl group, an optionally substituted heteroaryl group, an optionally substituted ketoalkyl, an optionally substituted amide, and an optionally substituted ketoheteroalkyl;
one of A or B is -L-G and the other is selected from among H and an optionally substituted C₁-C₆ alkyl; or
L is a bond, or an optionally substituted group selected from among C₁-C₆ alkylene, C₁-C₆ heteroalkylene, C₁-C₆ ketoalkylene, a monosaccharide, a disaccharide, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)—, —C(═NR¹⁰)N(R⁹)—(CH₂)ₙ—, and —NR⁹C(═NR¹⁰)N(R⁹)—(CH₂)ₙ—;
G is H, tetrazolyl, —CH₂—(O—CH₂—CH₂)_{q}—O—CH₃, —O—(CH₂—CH₂—O)_{q}—CH₃, —CH₂—(O—CH₂—CH₂)_{q}OH, —O—(CH₂—CH₂—O)_{q}H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H₃, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —(OP(═O)OH)ₙOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NO₂;
n is 1, 2, 3, or 4; x is 1, 2, or 3; y is 0, 1, or 2;
pharmaceutically acceptable salts, pharmaceutically acceptable N-oxides, pharmaceutically active metabolites, pharmaceutically acceptable pro drugs, or pharmaceutically acceptable solvates thereof; and
indicating a need to increase the amount of the drug subsequently administered to the subject for a level of the at least one fatty acid amide less than 50% of a pre-determined value.

26. The method of claim 1, wherein the at least one fatty acid amide is oleoylethanolamide.

27. The method of claim 1, wherein the at least one fatty acid amide is palmitoylethanolamide.

28. The method of claim 1, wherein the at least one fatty acid amide is stearoylethanolamide.

29. The method of claim 1, wherein the subject is a human.

30. The method of claim 1, wherein the subject is a non-human primate.

31. The method of claim 13, wherein the biological sample is plasma, saliva, whole blood, serum, or cerebrospinal fluid.

32. The method of claim 31, wherein the biological sample is plasma.

33. The method of claim 31, wherein the biological sample is whole blood.

34. The method of claim 31, wherein the biological sample is saliva.

35. The method of claim 24, wherein the drug is administered orally.

36. The method of claim 24, further comprising performing a diagnostic evaluation of the subject before and after administering the drug.

37. The method of claim 1, wherein the fatty acid amide hydrolase inhibitor is an alkylcarbamic acid aryl ester of Formula (VII): 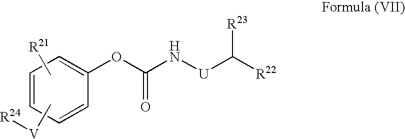
wherein:
U is a bond or CH₂;
V is a bond, CH₂, NH, or NR²⁵;
R²¹ is H, a carboxylic acid bioisostere, or a C₃-C₇ heterocycle;
R²² and R²³ are individually H, C₁-C₈ alkyl, (C₃-C₇ cycloalkyl), (C₁-C₄ alkyl(C₃-C₇ cycloalkyl)); or
R²² and R²³ together form a 3-, 4-, 5-, 6-, or 7-membered cycloalkyl or an oxygen containing heterocycloalkyl group;
R²⁴ is H, —(CO)R²⁵, —CO₂H, a carboxylic acid bioisostere, —(CO)NH₂, —(CO)NHR²⁵, —NH(CO)R²⁵, —NR(CO)R²⁷; or
R²¹ and R²⁴ together form an optionally substituted C₅-C₆ heterocycle;
R²⁵ and R²⁷ are independently selected from H or an alkyl group; and
pharmaceutically acceptable salts, pharmaceutically acceptable N-oxides, pharmaceutically active metabolites, pharmaceutically acceptable pro drugs, or pharmaceutically acceptable solvates thereof.

38. The method of claim 37, wherein the fatty acid amide hydrolase inhibitor is an alkylcarbamic acid aryl ester of Formula (IX) comprising: 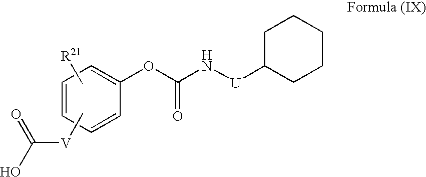
wherein:
U and V are individually a bond or CH₂;
R²¹ is H; and
pharmaceutically acceptable salts, pharmaceutically acceptable N-oxides, pharmaceutically active metabolites, pharmaceutically acceptable pro drugs, or pharmaceutically acceptable solvates thereof.

39. The method of clam 38, wherein the alkylcarbamic acid aryl ester of Formula (IX) is selected from: 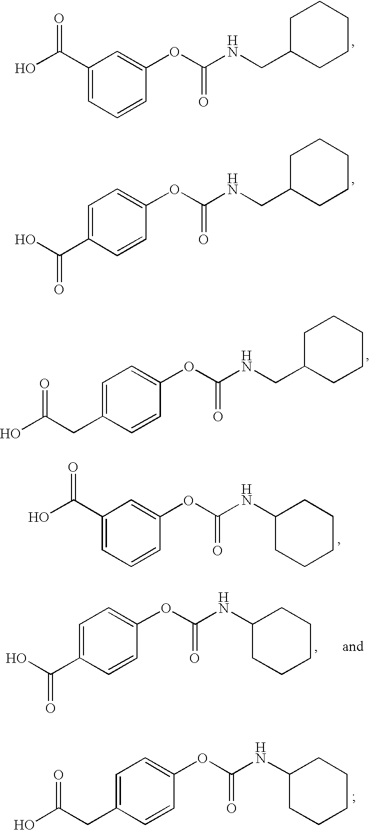
and pharmaceutically acceptable salts, pharmaceutically acceptable N-oxides, pharmaceutically active metabolites, pharmaceutically acceptable pro drugs, or pharmaceutically acceptable solvates thereof.

40. The method of claim 37, wherein the fatty acid amide hydrolase inhibitor is an alkylcarbamic acid aryl ester of Formula (VIII) comprising: 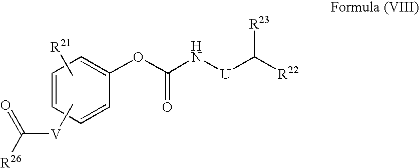
wherein:
U is a bond or CH₂;
V is NH or NR²⁵;
R²¹ is H;
R²² and R²³ together form a 6-membered cycloalkyl group;
R²⁵ and R²⁶ are individually an alkyl group; and
pharmaceutically acceptable salts, pharmaceutically acceptable N-oxides, pharmaceutically active metabolites, pharmaceutically acceptable pro drugs, or pharmaceutically acceptable solvates thereof.

41. The method of claim 40, wherein the alkylcarbamic acid aryl ester of Formula (VIII) is selected from: 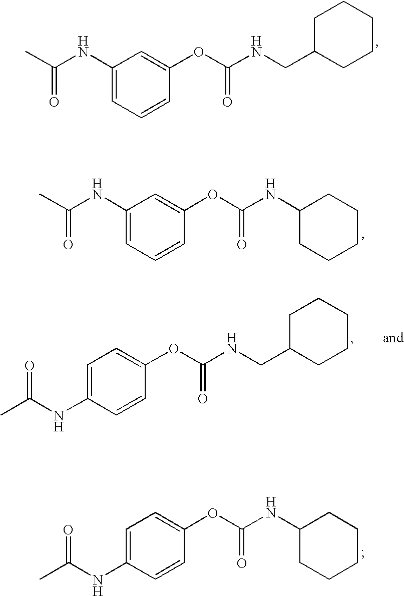
and pharmaceutically acceptable salts, pharmaceutically acceptable N-oxides, pharmaceutically active metabolites, pharmaceutically acceptable pro drugs, or pharmaceutically acceptable solvates thereof.

42. The method of claim 37, wherein the fatty acid amide hydrolase inhibitor is an alkylcarbamic acid aryl ester of Formula (VIII) comprising: 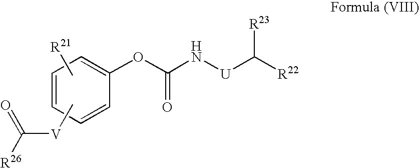
wherein:
U and V are individually a bond or CH₂;
R²¹ is H;
R²² and R²³ together form a 6-membered cycloalkyl group;
R²⁵ is an alkyl group;
R²⁶ is NH₂ or NHR²⁵; and
pharmaceutically acceptable salts, pharmaceutically acceptable N-oxides, pharmaceutically active metabolites, pharmaceutically acceptable pro drugs, or pharmaceutically acceptable solvates thereof.

43. The method of claim 42, wherein the alkylcarbamic acid aryl ester of Formula (VIII) is: 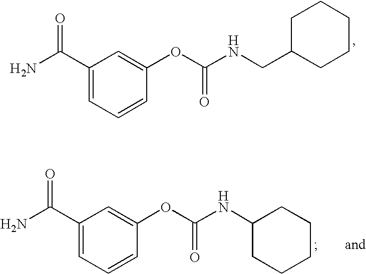
pharmaceutically acceptable salts, pharmaceutically acceptable N-oxides, pharmaceutically active metabolites, pharmaceutically acceptable pro drugs, or pharmaceutically acceptable solvates thereof.

44. The method of claim 37, wherein the fatty acid amide hydrolase inhibitor is an allycarbamic acid aryl ester of Formula (X): 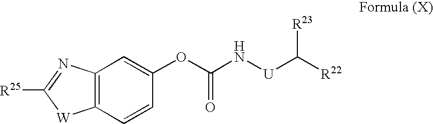
wherein:
U is a bond or CH₂;
R²² and R²³ together form a 6-membered cycloalkyl group;
W is O or S;
R²⁵ is H or an alkyl group; and
pharmaceutically acceptable salts, pharmaceutically acceptable N-oxides, pharmaceutically active metabolites, pharmaceutically acceptable pro drugs, or pharmaceutically acceptable solvates thereof.

45. The method of claim 44, wherein the alkylcarbamic acid aryl ester of Formula (XI) is selected from: 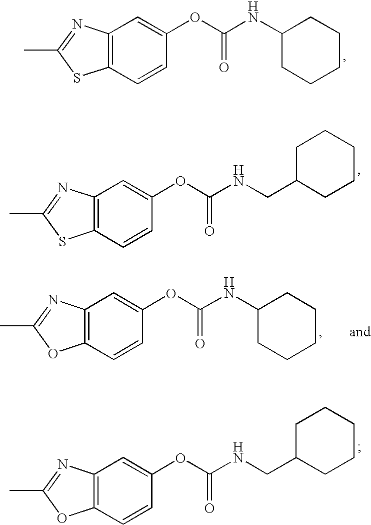
and pharmaceutically acceptable salts, pharmaceutically acceptable N-oxides, pharmaceutically active metabolites, pharmaceutically acceptable prodrugs, or pharmaceutically acceptable solvates thereof.

46. The method of claim 37, wherein the fatty acid amide hydrolase inhibitor is an alkylcarbamic acid aryl ester of Formula (XII) comprising: 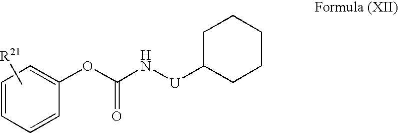
wherein:
U is a bond or CH₂;
R²¹ is a tetrazolyl group; and
pharmaceutically acceptable salts, pharmaceutically acceptable N-oxides, pharmaceutically active metabolites, pharmaceutically acceptable pro drugs, or pharmaceutically acceptable solvates thereof.

47. The method of claim 46, wherein the compound of Formula (XII) selected from: 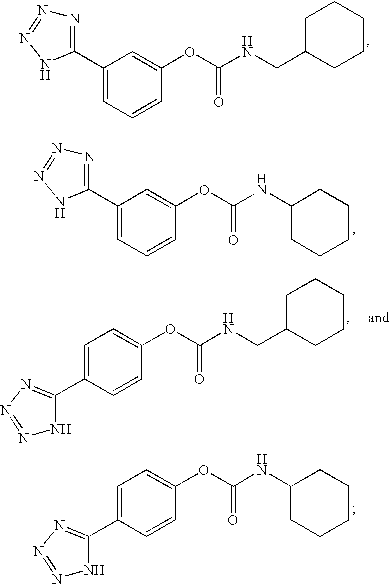
and pharmaceutically acceptable salts, pharmaceutically acceptable N-oxides, pharmaceutically active metabolites, pharmaceutically acceptable pro drugs, or pharmaceutically acceptable solvates thereof.

48. The method of claim 24, wherein the pain is selected from nociceptive pain, neuropathic pain, inflammatory pain, non-inflammatory pain, painful hemorrhagic cystitis, pain associated with the herpes virus, pain associated with diabetes, peripheral neuropathic pain, peri-operative pain, cancer pain, pain and spasticity associated with multiple sclerosis, central pain, deafferentiation pain, chronic nociceptive pain, stimulus of nociceptive receptors, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

49. The method of claim 1, wherein the indicating step comprises providing a physician with the level of the at least one fatty acid amide in the biological sample.

50. The method of claim 1, wherein the indicating step further comprises providing a physician with an interpretation of the level of the at least one fatty acid amide in the biological sample.

51. The method of claim 1, wherein the indicating step further comprises providing a physician with an interpretation to adjust a subsequent dose of the fatty acid amide hydrolase inhibitor.
